(19) United States
(12) Patent Application Publication
(10) Pub. No.: US 2023/0201270 A1
(43) Pub. Date: Jun. 29, 2023

(54) FIBROBLAST THERAPY FOR PREVENTION AND REVERSION OF ANEURYSMS

(71) Applicant: FIGENE, LLC, Houston, TX (US)

(72) Inventor: Thomas ICHIM, San Diego, CA (US)

(21) Appl. No.: 17/999,583

(22) PCT Filed: May 28, 2021
(86) PCT No.: PCT/US2021/070638

§ 371 Date: Nov. 22, 2022

Related U.S. Application Data
(60) Provisional application No. 63/031,782, filed on May 29, 2020.

(51) Int. Cl. A61K 35/33; A61K 45/06; A61K 9/00; C12N 5/077; A61P 9/14

(52) U.S. Cl. CPC A61K 35/33; A61K 9/0019; A61K 45/06; A61P 9/14; C12N 5/0656

(57) ABSTRACT

Embodiments of the disclosure include methods of inhibiting and/or reversing blood vessel degeneration in an individual by administering to the individual a therapeutically effective amount of a fibroblast cell population. In specific embodiments, the individual has one or more aneurysms. Also disclosed are methods of inhibiting development of aortic dissection and/or reversing blood flow abnormalities associated with aortic dissection. In specific cases the cells are CD73 -positive and/or CD56-positive.

This application claims priority to U.S. Provisional Patent Application Serial No. 63/031782, filed May 29, 2020, which is incorporated by reference herein in its entirety.

### TECHNICAL FIELD

Embodiments of the disclosure include at least the fields of cell biology, neurology, molecular biology, cell therapy, and medicine.

### BACKGROUND

There are two main types of arteries: elastic and muscular. Elastic arteries are large in nature (>1 cm diameter) while muscular ones are usually smaller (0.1-10 mm). In general, elastic arteries are close to the large pressure changes of the heart and therefore require elastic capabilities to buffer the pulse. The aorta is an example of an elastic artery and is capable of distension and elasticity which is required for it to be able to stretch as a response to each pulse of the heart. Another example of an elastic artery is the pulmonary artery, which delivers hypoxic blood to the lungs. The carotid, subclavian, and renal arteries are also considered elastic arteries. The tunica intima, or intima for short, is the innermost layer of an artery. It is made up of one layer of endothelial cells and is supported by an internal elastic lamina. The endothelial cells are in direct contact with the blood flow. The tunica media, or middle layer, of elastic arteries is characterized by the presence of numerous elastic lamella. The adventitia, or outermost layer of the wall of large blood vessels, carries vasa vasorum and nerves. The vasa vasorum is a network of small blood vessels that supply the walls of large blood vessels, such as elastic arteries. Connective tissue is usually present underneath elastic arteries.

In general, muscular arteries are medium size arteries which carry blood away from the larger elastic arteries. In muscular arteries, the tunica media is composed primarily of smooth muscle tissue. The muscular arteries contract, and the extent of contraction or relaxation is governed by endothelium-derived vasoactive substances such as nitric oxide, as well as by the nervous system. Although muscular arteries have some elastic fibers like elastic arteries, these are not organized into lamella.

The endothelium comprises the lining of blood vessels and is known to actively participate in numerous functions including secretion of coagulation and anti-coagulation factors [1], contraction and relaxation of the blood vessels by secretion of soluble factors [2], and recruitment of immunocytes [3] and stem cells [4] through expression of adhesion molecules. Many of the major diseases afflicting society are associated with endothelial dysfunction. For example, heart attack and stroke are associated with thrombotic states, usually as a result of hypercoagulation and/or lack of fibrinolysis. Ischemic heart failure is associated with poor collateralization and angiogenesis. Atherosclerosis, which causes the thickening of the blood vessels leading to ischemia, is caused by foam cell accumulation and progression to atheroma. Endothelial-dependent migration of monocytes and accessory cells is critical for development of atherosclerosis. Sepsis is caused by endothelial-dependent disseminated intravascular coagulation; the only drug for this condition which has demonstrated therapeutic benefit, recombinant activated protein C, acts on the endothelium [5]. Cancer is also associated with endothelium dysfunction in the sense that tumors are dependent on endothelium migration and angiogenesis for their growth and metastasis. Accordingly, controlling the endothelium and assuring its health is an important endeavor.

Endothelial damage can be caused by numerous factors. In addition to conditions which induce endothelial dysfunction, such as smoking, infections, and oxidative stress, endothelial dysfunction also occurs as a natural part of aging. For example, a recent study compared flow mediated dilation responses in healthy patients of various ages, free of cardiovascular risk factors. A statistically significant decline in vasodilatory response was observed to be positively correlated with age [6]. One possible explanation for age-related endothelial dysfunction is the decreased ability of the endothelium to secrete the vasoactive small molecule nitric oxide in response to appropriate stimuli. For example, Laurel et al. demonstrated inhibited exercise-induced nitric oxide production and flow mediated dilation in 28 aged (58 +/- 2 years old) healthy volunteers compared to 29 younger (25 +/- 1 years old) subjects [7]. Endothelium dysfunction has also been ascribed to low grade inflammation associated with aging. One useful marker of this is plasma levels of C Reactive Protein (CRP). Studies have demonstrated a positive correlation between age and plasma CRP [8], as well as CRP and presence of endothelial dysfunction [9]. Transgenic expression of CRP in mice leads to endothelial dysfunction, presumably through suppression of nitric oxide production and stimulation of macrophage infiltration into major blood vessels [10].

Endothelial damage is also associated with weakened blood vessels and smooth muscle cell apoptosis [12], as well as disorganization of the extracellular matrix. Certain conditions such as Marfan syndrome predispose individuals to weakening of blood vessels, though senescence and inflammation have been the cited cause of weakened blood vessels in the majority of patients. Weakening of blood vessels can lead to a variety of circulatory problems, including aneurysms and aortic dissection.

Aneurysms are blood-filled bulges in blood vessels caused by weakening of an artery or vein. Commonly aneurysms occur at the circle of Willis, located on the base of the brain, and in the aorta, although they can occur elsewhere. Bursting of the blood vessel causes death. Aneurysms appear either as small bubbles distending from the side of the blood vessel, known as saccular aneurysms, or as an expansion of the entire circumference of the blood vessel such that the vessel takes on a prolate spheroid shape, known as fusiform aneurysms. Dissecting aneurysms, like aortic dissections, are characterized longitudinal ripping, or dissecting, of the intimal layer of a blood vessel and subsequent hematoma formation in the area previously occupied by the tunica intima. The hematoma may cover significant portions of the lumen of the blood vessel, resulting in obstruction of blood flow.

Dysfunctional and damaged endothelium, which can result in weakened blood vessels and aneurysms, is known to be replenished by circulating endothelial precursor cells (EPC). It is known that such cells migrate to damaged arterial endothelium as a result of CXCR2 expression by EPCs which respond to CXCL1 or CXCL7 secreted by damaged endothelium [11].

Currently, the only therapeutic intervention for aneurysms and aortic dissection is surgical, which is associated with significant risk. Accordingly there is a need in the art for non-surgical methods of treating vascular degeneration and the specific consequences of vascular degeneration such as aortic dissection and aneurysms.

### BRIEF SUMMARY

The present disclosure is directed to systems, methods, and compositions for stimulation of blood vessel regeneration through administration of stem cells alone or in combination with agents capable of stimulating stem cells. The disclosure is related to the area of vascular biology. More particularly, the disclosure deals with methods of treating aneurysms or blood vessels prone to aneurysms.

In one embodiment, provided is a method of inhibiting and/or reversing blood vessel degeneration and/or promoting blood vessel regeneration in an individual, the method comprising the step of administering to the individual a therapeutically effective amount of a composition comprising a fibroblast cell population. The fibroblast cell population can be obtained from blood, placenta, bone marrow, amniotic fluid, amniotic membrane, circulating fibroblasts, testicular tissues, adipose tissue, exfoliated teeth, hair follicle, dermal tissue, side population cells, or a combination thereof. The fibroblast cell population can be autologous, allogeneic, xenogeneic, and a mixture thereof, and the fibroblast cell population can be derived from a donor younger in age than a recipient. In some embodiments, the fibroblasts are CD73-positive and/or CD56-positive fibroblasts. In some embodiments, the individual has at least one aneurysm. The aneurysm can be an aortic aneurysm or saccular aneurysm.

Provided in another embodiment is a method of treating one or more aneurysms in an individual, the method comprising the step of administering to the individual a therapeutically effective amount of a composition comprising one or more cell populations. The one or more cell populations can comprise fibroblast cells and stem cells. In some embodiments, at least one cell population comprises CD73-positive fibroblasts and/or CD56-positive fibroblasts administered individually or together. In some embodiments, approximately 1-500 million CD73-positive and/or CD56-positive fibroblast cells are administered to the individual. In some embodiments, approximately 750,000-1,250,000 CD73-positive and/or CD56-positive fibroblast cells are administered to the individual.

The composition of the method may also further comprises a fibroblast cell activator. The activator can be selected from the group consisting of erythropoietin, human chorionic gonadotrophin, parathyroid hormone, G-CSF, GM-CSF, valproic acid, thalidomide, sodium phenybutyrate, FGF-1, FGF-2, and combinations thereof. The fibroblast cell activator can increase the therapeutic activity of fibroblasts, and the therapeutic activity can include increased production of FGF-1, anti-inflammation, stimulation of tissue regeneration, or combinations thereof.

The method may further comprise mobilization of endogenous endothelial progenitor cells from the bone marrow. Mobilization can be achieved by administration of an agent selected from the group consisting of G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA)reductase inhibitors, small molecule antagonists of SDF-1, and combinations thereof. Mobilization can also be achieved by exposure to one or more conditions sufficient to mobilize endothelial progenitor cells. The one or more conditions can be selected from the group consisting of exercise, hyperbaric oxygen, autohemotherapy by ex vivo ozonation of peripheral blood, induction of SDF-1 secretion in an anatomical area outside of the bone marrow, and combinations thereof.

Also provided in one embodiment is a method of treating one or more aneurysms in an individual, the method comprising the step of administering to the individual a therapeutically effective amount of a composition comprising at least one fibroblast and/or stem cell population. The aneurysm can be an aortic aneurysm or a saccular aneurysm. Saccular aneurysms can be located in the brain. In some embodiments, the at least one fibroblast and/or stem cell population is administered at a concentration ranging from about 500,000 to about 200 million cells. In some embodiments, the at least one fibroblast and/or stem cell population is administered at a concentration ranging from about 750,000 to about 1,250,000 cells.

In some embodiments of the method, the fibroblast cell population inhibits progression or induces regression of an aneurysm. Inhibition of progression of an aneurysm can comprise suppression of progressive blood vessel weakening. Induction of regression of an aneurysm can comprise a reduction in circumference of an aneurysmic blood vessel.

In some embodiments, the at least one fibroblast and/or stem cell population expresses the markers C56, CD90, and/or CD105 and lacks expression of CD34 and/or CD45, and administration of the at least one fibroblast and/or stem cell population inhibits progression of an aortic aneurysm. In some embodiments, a CD56-positive fibroblast cell population is administered together with a mesenchymal or mesenchymal-like stem cell population, and wherein progression of an aortic aneurysm is inhibited. In some embodiments, the at least one fibroblast and/or stem cell population expresses the markers CD90 and CD105 and lacks expression of CD34 and CD45, and administration of the at least one fibroblast and/or stem cell population induces regression of an aortic aneurysm. In some embodiments, a CD73-positive fibroblast cell population is administered together with a mesenchymal or mesenchymal-like stem cell population, and regression of an aortic aneurysm is induced. In some embodiments, the at least one fibroblast and/or stem cell population expresses the markers CD56, CD90, and CD105 and lacks expression of CD34 and CD45, and administration of the at least one fibroblast and/or stem cell population inhibits progression of a saccular aneurysm. In some embodiments, a CD73-positive fibroblast cell population is administered together with a mesenchymal or mesenchymal-like stem cell population, and wherein progression of a saccular aneurysm is inhibited. In some embodiments, the at least one fibroblast and/or stem cell population expresses the markers CD56, CD90, and CD105 and lacks expression of CD34 and CD45, and administration of the at least one fibroblast and/or stem cell population induces regression of a saccular aneurysm. In some embodiments, a CD56-positive fibroblast cell population is administered together with a mesenchymal or mesenchymal-like stem cell population, and wherein regression of a saccular aneurysm is induced.

Provided in another embodiment is a method of inhibiting development of aortic dissection and/or reversing blood flow abnormalities associated with aortic dissection, the method comprising the step of administering to the individual a therapeutically effective amount of a composition comprising at least one fibroblast and/or stem cell population. In some embodiments, the at least one fibroblast and/or stem cell population is administered at a concentration ranging from about 500,000 to about 200 million cells. In some embodiments, the at least one fibroblast and/or stem cell population is administered at a concentration ranging from about 750,000 to about 1,250,000 cells.

In some embodiments of the method, administration of the at least one fibroblast and/or stem cell population strengthens the aorta intimal layer, thereby reducing the probability of tears in the intimal layer. In some embodiments of the method, administration of the at least one fibroblast and/or stem cell population reduces accumulation of basophilic ground substances and the extent of medial cystic necrosis. In some embodiments of the method, administration of the at least one fibroblast and/or stem cell population restores substantially normal blood flow.

In some embodiments, the at least one fibroblast and/or stem cell population expresses the markers CD56, CD90, and CD105 and lacks expression of CD34 and CD45, and administration of the at least one fibroblast and/or stem cell population inhibits development of aortic dissection. In some embodiments, a CD56-positive fibroblast cell population is administered together with a mesenchymal or mesenchymal-like stem cell population, and wherein development of aortic dissection is inhibited. In some embodiments, the at least one fibroblast and/or stem cell population expresses the markers CD56, CD90, and CD105 and lacks expression of CD34 and CD45, and administration of the at least one fibroblast and/or stem cell population reverses blood flow abnormalities. In some embodiments, a CD73-positive stem cell population is administered together with a mesenchymal or mesenchymal-like stem cell population, and blood flow abnormalities are reversed.

The fibroblast cells provided in the methods of the disclosure can be obtained from cord blood, placenta, Wharton’s jelly, circulating peripheral blood, adipose tissue derived, exfoliated teeth, hair follicle, dermis, menstrual blood, endometrium, amnion, amniotic fluid, or a combination thereof. The stem cells of the methods can be selected from the group consisting of hematopoietic stem cells, embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells, side population stem cells, and combinations thereof.

In some embodiments, the fibroblast cell population is administered intravenously. The fibroblast cell population can be administered once every other day and/or once every other day over the course of 7 days. In embodiments with one or more cell populations, the one or more cell populations can be administered in sequence or at the same time. Administration of the fibroblast cell population can inhibit inflammation and/or accelerates re-endothelialization. Administration of the cell populations of the disclosure can also induce an environment conducive for blood vessel regeneration, restoration of blood vessel function, and/or reversal of blood vessel degeneration. The environment can be associated with reduced fibrosis, enhanced growth factor production, and stimulation of cellular proliferation. Blood vessel regeneration, restoration of blood vessel function, and/or reversal of blood vessel degeneration can extend the life of a mammal as a result of appropriate production of anticoagulating/clotting factors, control of angiogenesis, appropriate revascularization of injured tissue, decrease in age-related atherosclerosis, and prevention of loss of anti-thrombotic activity of endothelium associated with age.

In some embodiments, one or more additional therapeutic agents and/or conditions are administered in combination with the fibroblast cell population, the one or more cell populations, or the at least one fibroblast and/or stem cell population. The one or more additional therapeutic agents may be capable of: a) stimulating fibroblast integration into the blood vessels; b) augmenting regenerative activity of endogenous and/or exogenous fibroblasts, whether endogenous or exogenous; c) mobilizing endothelial progenitor cells; d) stimulating smooth muscle cell proliferation; e) inducing nitric oxide activity; or f) a combination thereof.

Agents capable of stimulating fibroblast cell integration into parts of blood vessels can be selected from the group consisting of a matrix metalloprotease inhibitor, an antioxidant, a chemoattractant, and combinations thereof. Agents capable of stimulating fibroblast regenerative cell activity can be selected from the group consisting of erythropoietin, human chorionic gonadotrophin, parathyroid hormone, G-CSF, GM-CSF, valproic acid, thalidomide, sodium phenybutyrate, FGF-1, FGF-2, and combinations thereof. Agents capable of mobilizing endothelial progenitor cells can be selected from the group consisting of G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, and combinations thereof. Conditions capable of mobilizing endothelial progenitor cells can comprise exposure to one or more conditions sufficient to mobilize endothelial progenitor cells. Conditions can be selected from the group consisting of exercise, hyperbaric oxygen, autohemotherapy by ex vivo ozonation of peripheral blood, induction of SDF-1 secretion in an anatomical area outside of the bone marrow, and combinations thereof. Agents capable of stimulating smooth muscle proliferation can be selected from the group consisting of PDGF-1, PDGF-BB, BTC-GF, estradiol, and combinations thereof. Agents inductive of nitric oxide activity can be selected from the group consisting of lipoteichoic acid, cinnamic acid, resveratrol, FGF, and combinations thereof.

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Brief Summary, Detailed Description, Claims, and

### BRIEF DESCRIPTION OF THE DRAWINGS

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

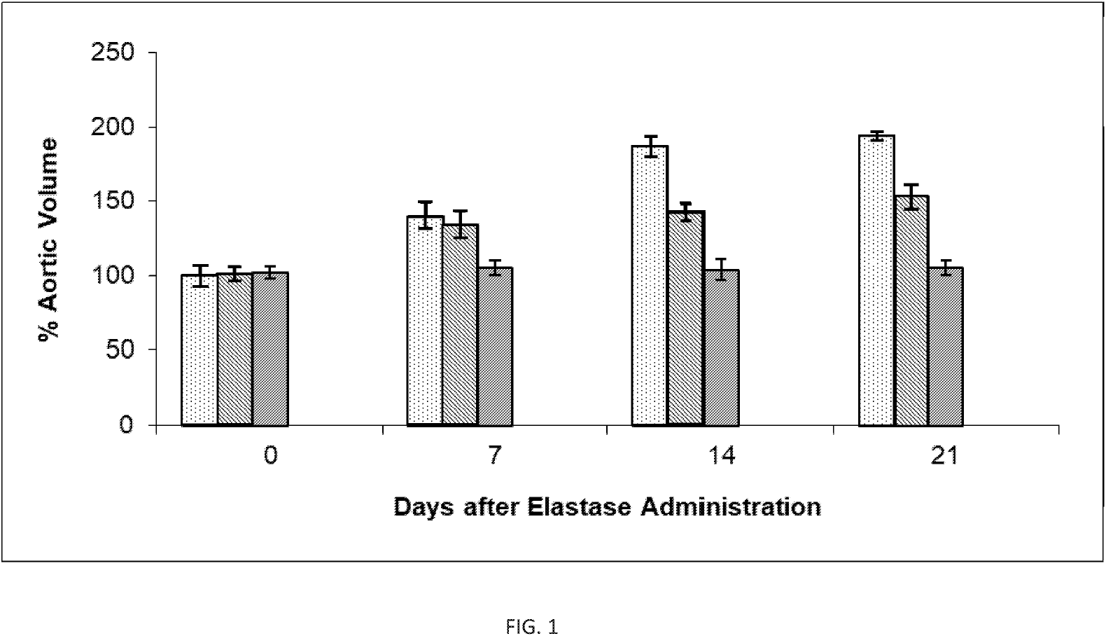

FIG. 1 shows the percent aortic volume in a mouse model for aneurysm. Animals receiving saline control (far left bar in groupings of three bars) had a progressive increase in aortic diameter; animals receiving CD73- fibroblasts (middle bar in groupings of three bars) possessed a decrease in expansion of aneurysm; and animals receiving CD73+ fibroblasts (far right bar in groupings of three bars) possessed the highest amount of aneurysm inhibitory activity. Each bar represents 8 animals.

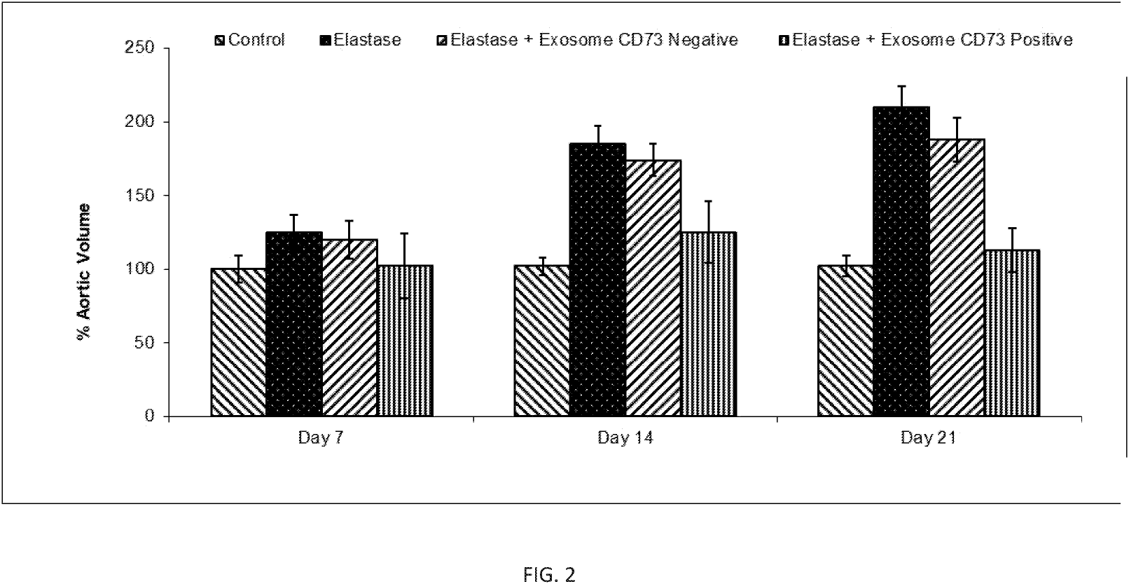

FIG. 2 shows percent aortic volume in a mouse model for aneurysm. The animals received control, elastase, elastase with exosomes from CD73-negative fibroblasts, and elastase with exosomes from CD73-positive fibroblasts.

### DETAILED DESCRIPTION

### I. Exemplary Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined. The terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. The term “about” is used according to its plain and ordinary meaning in the area of cell and molecular biology to indicate that a value includes the standard deviation of error for the device or method being employed to determine the value. The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment.

Furthermore, the particular features, structures, or characteristics may be combined in any suitable manner in one or more embodiments. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

As used here, “blood vessel degeneration” refers to the initiation and progression of various pathological processes which contribute to the development of blood vessel disorders, including weakening of the blood vessels and aneurysm formation.

As used herein, “cell culture” means conditions wherein cells are obtained (e.g., from an organism) and grown under controlled conditions (“cultured” or grown “in culture”) outside of an organism. A primary cell culture is a culture of cells taken directly from an organism (e.g., tissue cells, blood cells, cancer cells, neuronal cells, fibroblasts, etc.). Cells are expanded in culture when placed in a growth medium under conditions that facilitate cell growth and/or division. The term “growth medium” means a medium sufficient for culturing cells. Various growth media may be used for the purposes of the present disclosure including, for example, Dulbecco’s Modified Eagle Media (also known as Dulbecco’s Minimal Essential Media) (DMEM), or DMEM-low glucose (also DMEM-LG herein). DMEM-low glucose may be supplemented with fetal bovine serum (e.g., about 10% v/v, about 15% v/v, about 20% v/v, etc.), antibiotics, antimycotics (e.g., penicillin, streptomycin, and/or amphotericin B), and/or 2-mercaptoethanol. Other growth media and supplementations to growth media are capable of being varied by the skilled artisan. The term “standard growth conditions” refers to culturing cells at 37° C. in a standard humidified atmosphere comprising 5% CO₂. While such conditions are useful for culturing, it is to be understood that such conditions are capable of being varied by the skilled artisan who will appreciate the options available in the art for culturing cells. When cells are expanded in culture, the rate of cell proliferation is sometimes measured by the amount of time needed for the cells to double in number. This is referred to as doubling time.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

As used herein, “conditioned medium” describes medium in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. In this example, the medium containing the cellular factors is conditioned medium.

The term “compatible,” as used herein, means that the components of pharmaceutical compositions are capable of being commingled with the cells of the present disclosure and/or with other components of the pharmaceutical compositions, in a manner such that the desired pharmaceutical efficacy is not substantially impaired.

“Differentiation” (e.g., cell differentiation) describes a process by which an unspecialized (or “uncommitted”) or less specialized cell acquires the features (e.g., gene expression, cell morphology, etc.) of a specialized cell, such as a nerve cell or a muscle cell for example. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. In some embodiments of the disclosure, “differentiation” of fibroblasts to other cell types is described. This process may also be referred to as “transdifferentiation”.

As used herein, “dedifferentiation” refers to the process by which a cell reverts to a less specialized (or less committed) position within the lineage of a cell. As used herein, the lineage of a cell defines the heredity of the cell. The lineage of a cell places the cell within a hereditary scheme of development and differentiation. Within the context of the current disclosure, “dedifferentiation” may refer to fibroblasts acquiring more “immature” associated markers such as OCT4, NANOG, CTCFL, ras, raf, SIRT2, and SOX2. Additionally, “dedifferentiation” may mean acquisition of functional properties such as enhanced proliferation activity and/or migration activity towards a chemotactic gradient. In some embodiments fibroblasts may be “dedifferentiated” by treatment with various conditions, subsequent to which they are “differentiated” into other cell types.

“Fibroblasts” refer to a cell, progenitor cell, or differentiated cell and include isolated fibroblast cells or population(s) thereof capable of proliferating and differentiating into ectoderm, mesoderm, or endoderm. In some embodiments, fibroblasts are utilized in an autologous, allogenic, or xenogenic manner. As used herein, placental and adult-derived cellular populations are included in the definition of “fibroblasts.” In the context of the present disclosure, fibroblast cells may be derived through means known in the art from sources including at least foreskin, ear lobe skin, bone marrow, cord blood, placenta, amnion, amniotic fluid, umbilical cord, embryos, intraventricular cells from the cerebral spinal fluid, circulating fibroblast cells, mesenchymal stem cell associated cells, germinal cells, adipose tissue, exfoliated tooth-derived fibroblasts, hair follicle, dermis, skin biopsy, nail matrix, parthenogenically-derived fibroblasts, fibroblasts that have been reprogrammed to a dedifferentiated state, side population-derived fibroblasts, fibroblasts from plastic surgery-related by-products, and the like.

The terms “fibroblast-derived products” or “fibroblast-produced therapeutic factors” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

“Pharmaceutically acceptable carrier,” as used herein, includes one or more compatible solid or liquid filler diluents or encapsulating substances that are suitable for administration to a human or other animal. In the present disclosure, the term “carrier” thus denotes an organic or inorganic ingredient, which can be natural or synthetic, and with which the molecules of the disclosure are combined to facilitate application or administration. The carrier must also be compatible with the agent used to produce a desired result or exert a desired influence on the particular condition being treated.

As used herein, “regenerative activities” include but are not limited to therapeutic functions, stimulation of angiogenesis, inhibition of inflammation, and/or augmentation of tissue self-renewal, for example in part through activation of endogenous and/or exogenous stem and/or progenitor cells. Regenerative activities include the promotion of angiogenesis, suppression of inflammation, differentiation into blood vessel cells and/or smooth muscle cells, and/or secretion of growth factors such as IGF-1, EGF-1, FGF-2, VEGF, FGF-11, PDGF, HGF, and/or angiopoietin. In a particular embodiment, fibroblasts having one or more regenerative activities are isolated for one or more specific markers and subsequently transfected with one or more genes capable of endowing various therapeutic functions. Genes useful for stimulation of regenerative activities such as augmentation of hematopoietic activity include interleukin (IL)-3, IL-12, IL-23, GM-CSF, and/or TPO to stimulate proliferation of hematopoietic stem cells, for example. Other useful genes include IL-35, wherein in at least some cases IL-35 transfection allows for generation of cells possessing anti-inflammatory and angiogenic T regulatory cell activity, and the cells possessing T regulatory cell activities include cells expressing the transcription factor FoxP3, as an example.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition of any kind. The subject may have a disease or be suspected of having a disease. The subject may be asymptomatic. The subject may be of any gender, age, or race. The subject may be of a certain age, such as at least 30, 35, 40, 45, 50, 55, 60, 65, 70, 75, 80, 85, 90, 95, or 100 or more.

As used herein, the phrase “subject in need thereof” or “individual in need thereof” refers to a subject or individual, as described infra, that suffers or is at a risk of suffering (e.g, pre-disposed such as genetically pre-disposed, or subjected to environmental conditions that pre-dispose, etc.) from the diseases or conditions listed herein (e.g, aneurysms).

As used herein, the term “therapeutically effective amount” is synonymous with “effective amount”, “therapeutically effective dose”, and/or “effective dose” refers to an amount of an agent sufficient to produce a desired result or exert a desired influence on the particular condition being treated. In some embodiments, a therapeutically effective amount is an amount sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. Effective amount can also mean the amount of a compound, material, or composition comprising a compound of the present disclosure that is effective for producing some desired effect, e.g., treating or preventing aneurysms and/or blood vessel degeneration. For instance, in some embodiments, the effective amount refers to the amount of fibroblasts and/or fibroblast conditioned media that can promote blood vessel regeneration, restore blood vessel function, reverse blood vessel degeneration, and/or treat or prevent aneuyrsms in animals and humans. The effective amount may vary depending on the organism or individual treated.

The appropriate effective amount to be administered for a particular application of the disclosed methods can be determined by those skilled in the art, using the guidance provided herein. For example, an effective amount can be determined experimentally using various techniques and/or extrapolated from in vitro and in vivo assays including dose escalation studies. Various concentrations of an agent may be used in preparing compositions incorporating the agent to provide for variations in the age of the patient to be treated, the severity of the condition, and/or the duration of the treatment and the mode of administration. One skilled in the art will recognize that the condition of the individual can be monitored throughout the course of therapy and that the effective amount of a compound or composition disclosed herein that is administered can be adjusted accordingly. Further, one of skill in the art recognizes that an amount may be considered effective even if the medical condition is not totally eradicated but improved partially. For example, the medical condition may be halted or reduced or its onset delayed, a side effect from the medical condition may be partially reduced or completed eliminated, and so forth.

As used herein, the terms “treatment,” “treat,” or “treating” refers to intervention in an attempt to alter the natural course of the individual or cell being treated, and may be performed either for prophylaxis or during the course of pathology of a disease or condition, such as for example aneurysms and/or blood vessel degeneration. Treatment may serve to accomplish one or more of various desired outcomes, including, for example, preventing occurrence or recurrence of disease, alleviation of symptoms, and diminishment of any direct or indirect pathological consequences of the disease, preventing disease spread, lowering the rate of disease progression, amelioration or palliation of the disease state, and remission or improved prognosis, and/or producing some desired effect, e.g., blood vessel regeneration, restoration of blood vessel function, and/or reversal of blood vessel degeneration, suppression of progressive blood vessel weakening, and/or reduction in circumference of an aneurysmic blood vessel.

### II. General Embodiments

The present disclosure provides means of inhibiting and/or treating aneurysms and other degenerated blood vessels through administration of a fibroblast cell population and/or fibroblast-produced therapeutic factors. Fibroblast-produced therapeutic factors can include growth media conditioned by fibroblasts and/or exosomes, apoptotic bodies, or microvesicles produced by fibroblasts, for example. In one embodiment, vessel reactivity, or vasodilatory response to nitric oxide, is increased through administration of fibroblasts and/or fibroblast produced factors. Other embodiments include regeneration of vessels prone to aneurysms, repairing vessel aneurysms, or accelerating endothelialization after stent placement. Also provided are methods of rejuvenating properties of vessels associated with physiological health, including appropriate production of anti-coagulating/clotting factors, control of angiogenesis, and appropriate revascularization of injured tissue.

Embodiments of the disclosure include methods of inhibiting and/or reversing the process of blood vessel degeneration through administration of a fibroblast cell population. Specifically provided is the unexpected ability of systemically administered fibroblasts to benefit vascular function. In some embodiments, the fibroblast cell population acts as an immune modulator to prevent inflammation in blood vessels, which suppresses weakening of blood vessels and inhibits and/or reverses the process of blood vessel degeneration. As used herein, “blood vessel degeneration” means the initiation and progression of various pathological processes which contribute to the development of blood vessel disorders, including weakening of the blood vessels and aneurysm formation. In some cases, aneurysms form as a result of weakening of the supporting structures holding the blood vessel together due to inflammation. Inflammation can activate MMPs, which cause cleavage and deterioration of blood vessels. Fibroblasts can inhibit inflammation and accelerate re-endothelialization after damage to blood vessels.

In specific embodiments, a fibroblast cell population is used as a therapy to impede progression of vascular aneurysms. Previous studies have demonstrated the involvement of matrix metalloproteases (MMP) in dilation of vascular aneurysms [13]. In fact, MMP inhibitors have been proposed for clinical trials in patients with abdominal aortic aneurysms [14]. Studies have also demonstrated that various stem cells including hematopoietic [15, 16] and mesenchymal [17] stem cells express high levels of MMPs. However, administration of fibroblast cells, though similar to various stem cells, can contribute to regression of blood vessel disorders such as aneurysms rather than progression of the pathological processes contributing to development of blood vessel disorders.

In some embodiments, fibroblasts are administered together with hematopoietic stem cells to an individual in need thereof. In some embodiments, fibroblasts are administered together with various types of stem cells, committed progenitor cells, and/or differentiated cells. The fibroblasts may be reprogrammed or isolated as a side population of cells, as described elsewhere herein. The stem cells may be selected from the group consisting of embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue-derived stem cells, exfoliated teeth-derived stem cells, hair follicle stem cells, dermal stem cells, parthenogenically-derived stem cells, reprogrammed fibroblasts, side population fibroblasts, and a combination thereof. The individual can have weakened blood vessels. The individual may have weakened blood vessels for any reason. Blood vessels in the individual may be weakened due to endothelia damaged as a result of increased age, smoking, infections, and/or oxidative, or as a result of smooth muscle cell apoptosis, for example.

According to some embodiments, fibroblasts are incubated with one or more growth factors (i.e. mitogenic compounds) under growth conditions suitable to allow for proliferation and to prepare the fibroblasts for differentiation into blood vessel-associated cells. Blood vessel-associated cells can include smooth muscle cells, endothelial cells, nerve cells, and pericytes. In further embodiments, fibroblasts are incubated with one or more differentiation factors and/or one or more inducing agents, and optionally, one or more growth factors, under conditions suitable to allow for differentiation, and optionally, proliferation, of a variety of cell types which prevent and/or reverse blood vessel degeneration by stimulating regenerative processes. Known compounds that function as both growth factors and differentiation inducers are recognized by those of skill in the art. For example, growth factors can include but are not limited to M-CSF, IL-6, LIF, and IL-12, and compounds functioning as growth factors and/or differentiation inducers can include but are not limited to lipopolysaccharide (LPS), phorbol 12-myristate 13-acetate (PMA), stem cell growth factor, human recombinant interleukin-2 (IL-2), IL-3, epidermal growth factor (EGF), b-nerve growth factor (bNGF), recombinant human vascular endothelial growth factories₁₆₅ isoform (VEGF₁₆₅), and/or hepatocyte growth factor (HGF). Doses of growth and/or differentiation factors used to induce proliferation of fibroblasts and increase susceptibility of fibroblasts to differentiation include but are not limited to from about 0.5 ng/ml-10.0 µg/ml or 1.0 µg/ml LPS, from about 1-160 nM or 3 nM PMA, from about 500-2400 units/ml or 1200 ng/ml bNGF, from about 12.5-100 ng/ml or 50 ng/ml VEGF, from about 10-200 ng/ml or 100 ng/ml EGF, and from about 25-200 ng/ml or 50 ng/ml for HGF.

In some embodiments, reduction in TNF-alpha secretion in cells of the individual is utilized prior to, together with, and/or subsequent to administration of a fibroblast cell population, endothelial progenitor cells, and/or stem cells. Suitable inhibitors of TNF-alpha secretion include but are not limited to: cycloheximide [31], auranofin, sodium aurothiomalate, and triethyl gold phosphine [32], lipoxygenase inhibitors [33-36], ethanol [37, 38], Leukotriene B4 [39], interleukin-4 [40], interleukin-13 [41], polymyxin B [42, 43], bile acids [44], interleukin-6 [45], lactulose [46], oxpentifylline [47], mometasone [48], glucocorticoids [49], colchicine [50], chloroquine [51], FK-506 [52, 53], cyclosporine [54], phosphodiesterase inhibitors such as vinpocetine, milrinone, CI-930, rolipram, nitroquazone, zaprinast [55], synthetic lipid A [56, 57], amrinone [58], N-acetylcysteine [59], dithiocarbamates and metal chelators [60], exosurf synthetic surfactant [61], dehydroepiandrosterone [62], delta-tetrahydrocannabinol [63, 64], phosphatidylserine [65], TCV-309, a PAF antagonist [66], thalidomide [67-69], cytochrome p450 inhibitors such as Metyrapone and SKF525A [70], cytochalasin D [71], ketamine [72], TGF-beta [73], interleukin-10 [74], pentoxifylline [75], BRL 61,063 [76], calcium antagonists such as dantrolene, azumolene, and diltiazem [77], curcumin [78], kappa-selective opioid agoinst U50,488H (trans-3,4-dichloro-N-methyl-N-[7-(1-pyrrolidinyl)cyclohexyl]benzene-acetamide methanesulfonate) [79], alendronate [80], alkaloids such as fangchinoline and isotetrandrine [81], plant alkaloids such as tetrandrine [82], sulfasalazine [83], epinephrine [84], BMS-182123 [85], adenosine [86, 87], E3330 [88], nicotine [89, 90], IVIG [91, 92], cardiotrophin-1 [93], KB-R7785 [93], CGRP [94], ligustrazine [95], dexanabinol [96], iloprost [97], activated protein C [98], growth hormone [99], spermine [100], FR-167653 [101], gm-6001 [102], estradiol [103], aspirin [104], and amiodarone [105].

In some embodiments, TNF-alpha activity inhibitors are administered systemically and/or locally to suppress inflammation and allow for enhancement of therapeutic effects of cells and/or regenerative factors administered intrathecally, intramuscularly, intravenously, or intracerebrally. Local administration can be intradiscal, intramuscular in proximity to an aneurysm, on the vascular bed surrounding an aneurysm, and/or in the interstitial fluid surrounding an aneurysm. Suitable inhibitors of TNF-alpha activity include but are not limited to: ibuprofen and indomethacin [106], Nedocromil sodium and cromolyn (sodium cromoglycate) [107], spleen derived factors [108], pentoxifylline [109-111], the 30 kDa TNF-alpha inhibitor [112], NG-methyl-L-arginine [113], antibodies directed against the core/lipid A [114], dexamethasone [115], chlorpromazine [116], activated alpha 2 macroglobulin [117], serum amyloid A protein [118], neutrophil derived proteolytic enzymes [119], phentolamine and propranolol [120], leukotriene inhibitors [121], nordihydroguaiaretic acid [122], genistein [123], butylated hydroxyanisole [124], CNI-1493 [125], quercetin [126], gabexate mesylate [127], SM-12502 [128], monoclonal nonspecific suppressor factor (MNSF) [129], pyrrolidine dithiocarbamate (PDTC) [130], and aprotinin [131].

It is known that under certain conditions, fibroblasts are capable of producing interleukin-1 and/or other inflammatory cytokines [132]. Embodiments of the present disclosure include fibroblasts treated by gene editing of IL-1 and/or other inflammatory mediators to prevent expression of one or more inflammatory cytokines by fibroblasts after administration. In some embodiments, TNF-alpha and inflammatory mediators are suppressed in fibroblasts, including those administered to the brain, systemically, intramuscularly, intravenously, intra-arterially, and in proximity to the location of an aneurysm. However, the fibroblasts can be pretreated with TNF-alpha to induce expression of growth and/or proliferation factors, as described in the art and incorporated by reference; specifically, it has been shown that TNF-alpha is capable of stimulating production of agents such as IGF-1, HGF-1, and FGF-2, which have been shown to be involved in stimulating tissue regeneration [133, 134].

### III. Cells of the Disclosure

Certain aspects of the present disclosure relate to administration of fibroblasts together with hematopoietic stem cells to an individual in need thereof. In some embodiments, fibroblasts are administered together with various types of stem cells, committed progenitor cells, and/or differentiated cells. The fibroblasts may be reprogrammed or isolated as a side population of cells, as described elsewhere herein. The stem cells may be selected from a group consisting of embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue-derived stem cells, exfoliated teeth-derived stem cells, hair follicle stem cells, dermal stem cells, parthenogenically-derived stem cells, reprogrammed stem cells, and side population stem cells.

### Fibroblasts

Fibroblasts of the present disclosure are administered together with other cell types to inhibit and/or treat aneurysms and other degenerated blood vessels. In some embodiments, the fibroblasts are regenerative fibroblasts. Regenerative fibroblast cells may be generated by culturing fibroblasts under conditions sufficient to generate a regenerative fibroblast cell. In some embodiments, the fibroblast cells can provide a tissue with regenerative activity. In some embodiments, the method includes culturing the population of fibroblast regenerative cells under conditions that support proliferation of the cells. In additional embodiments, the fibroblast cells may be cultured under conditions that form tissue aggregate bodies. In some embodiments, the fibroblast cells are used to create other cell types for tissue repair or regeneration. Generation of fibroblasts has been described previously in the art and is incorporated herein. Generally, fibroblasts are harvested from dissociated tissues of interest. Accomplishing the dissociation process can be performed mechanically or by treatment of tissue with enzymes, such as dispase or collagenase, which releases cells from the tissues. Subsequently, single cell fibroblasts are isolated based on plastic adherence and then allowed to expand and proliferate in liquid media.

Specific desirable properties of fibroblast cells of the present disclosure are the ability to increase endothelial function; suppress inflammation; induce neo-angiogenesis; prevent atrophy; differentiate into functional tissue; induce local resident stem and/or progenitor cells to proliferate through secretion of soluble factors or membrane bound activities; induce generation of T regulatory cells; inhibit Th1 and Th17 immunity; and/or promote immunological tolerance. In some embodiments, the fibroblast cells are selected for the ability to differentiate into blood vessel-associated cells prevent and/or reverse blood vessel degeneration by stimulating regenerative processes.

When selecting fibroblast cells, several factors must be taken into consideration, including the ability for ex vivo expansion without loss of therapeutic activity, ease of extraction, general potency of activity, and potential for adverse effects. Ex vivo expansion ability of fibroblasts can be measured using typical proliferation and colony assays known to one skilled in the art, for example, while identification of therapeutic activity may utilize one or more functional assays that test biological activities correlated with one or more therapeutic goals.

In some embodiments, assessment of therapeutic or regenerative activity is performed using surrogate assays which detect one or more markers associated with a specific therapeutic activity. In some embodiments, assays used to identify therapeutic activity of fibroblast cell populations include evaluation of the production of one or more factors associated with desired therapeutic activity. In some embodiments, evaluation of the production of one or more factors to approximate therapeutic activity in vivo includes identification and quantification of the production of FGF, VEGF, angiopoietin, a combination thereof, or other angiogenic molecules such as HGF-1, PDGF-1, and/or IL-35 that may be used to serve as a guide for approximating therapeutic activity in vivo. Other therapeutic activities are associated with cytokine activity, which can be quantified. For example, to assess the Treg stimulating activity of the cells, IL-10 production may be quantified; to assess the regenerative activity of the cells, GDF-11 may be quantified; and to assess the anti-apoptotic activity of the cells, suppression of caspase 3 activation may be assessed.

In one embodiment, fibroblast cells are collected from an autologous patient, expanded ex vivo, and reintroduced into the patient at a concentration and frequency sufficient to cause therapeutic benefit. In other cases, the expanded cells are allogeneic or xenogenic with respect to a recipient individual. In some embodiments, fibroblasts of the present disclosure are used as precursor cells that differentiate following introduction into an individual. In some embodiments, fibroblasts are subjected to differentiation into a different cell type (e.g., a hematopoietic cell) prior to introduction into the individual.

In some embodiments, the fibroblast cells used in combination with other cell types are capable of proliferating and differentiating into ectoderm, mesoderm, or endoderm. In some embodiments, the enriched population of fibroblast cells are about 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 micrometers in size.

In some embodiments, the fibroblast cells are expanded and/or differentiated in culture using one or more cytokines, chemokines and/or growth factors prior to administration to an individual in need thereof. The agent capable of inducing fibroblast expansion can be selected from the group consisting of TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, a member of the bone morphogenic protein family, and a combination thereof. The agent capable of inducing fibroblast differentiation can be selected from the group consisting of HGF, BDNF, VEGF, FGF1, FGF2, FGF4, FGF20, and a combination thereof.

In some embodiments, the cultured fibroblast cells express proteins characteristic of normal fibroblasts including the fibroblast-specific marker, CD90 (Thy-1), a 35 kDa cell-surface glycoprotein, and the extracellular matrix protein, collagen. In some embodiments, fibroblast cells express at least one of Oct-4, Nanog, Sox-2, KLF4, c-Myc, Rex-1, GDF-3, LIF receptor, CD105, CD117, CD344 or Stella markers. In some embodiments, the fibroblasts express CD73, CD90, and/or CD105. In some embodiments, fibroblasts of the present disclosure express telomerase, Nanog, Sox2, β-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-α, HGF, c-MET, α-1-Antriptrypsin, Ceruloplasmin, AFP, PEPCK 1, BDNF, NT-⅘, TrkA, BMP2, BMP4, FGF2, FGF4, PDGF, PGF, TGFα, TGFβ, and/or VEGF. In some embodiments, the fibroblast cells do not produce one or more of CD14, CD31, CD34, CD45, CD117, CD141, HLA-DR, HLA-DP, HLA-DQ, or a combination thereof. In further embodiments, the fibroblast regenerative cell has enhanced expression of GDF-11 as compared to a control. In still further embodiments, the fibroblast cells express CD73, which is indicative of fibroblast cells having regenerative activity.

In some embodiments, fibroblast cells do not express at least one of MHC class I, MHC class II, CD45, CD13, CD49c, CD66b, CD73, CD105, or CD90 cell surface proteins. In some embodiments, the method optionally includes the step of depleting cells expressing stem cell surface markers or MHC proteins from the cell population, thereby isolating a population of stem cells. In some embodiments, the cells to be depleted express MHC class I, CD66b, glycophorin a, and/or glycophorin b. In some embodiments, fibroblast cells are isolated and expanded and possess one or more markers selected from the group consisting of CD10, CD13, CD44, CD73, CD90, CD141, PDGFr-α, HLA-A, HLA-B, HLA-C, and a combination thereof.

In some cases, fibroblast cells are obtained from a biopsy, and the donor providing the biopsy may be either the individual to be treated (autologous), or the donor may be different from the individual to be treated (allogeneic). In some embodiments, the fibroblast cells are xenogenic with respect to a recipient individual. In some embodiments wherein allogeneic fibroblast cells are utilized for an individual, the fibroblast cells may come from one or a plurality of donors. In some embodiments fibroblasts are used from young (less than 25 years old) donors. In some embodiments, steps are taken to protect allogeneic or xenogenic cells from immune-mediated rejection by the recipient. Steps include encapsulation, co-administration of an immune suppressive agent, transfection of said cells with immune suppressory agent, or a combination thereof. In some embodiments, tolerance to the cells is induced through immunological means. In some embodiments, fibroblasts are transfected with genes to allow for enhanced growth and to overcome the Hayflick limit. Subsequent to derivation by biopsy, the fibroblasts are expanded in culture using standard cell culture techniques.

Biopsy is performed by extracting tissue, usually 0.1 grams to 10 grams. In some situations biopsy may be performed using a biopsy gun or alternatively using a scalpel. In one embodiment, biopsies are from skin tissue (dermis and epidermis layers) from a subject’s post-auricular area. In one embodiment, the starting material is composed of three 3-mm punch skin biopsies collected using standard aseptic practices, though the methods disclosed herein for preparing a skin biopsy tissue would apply equally to biopsies of other tissue types. The biopsies are collected by the treating physician, placed into a vial containing sterile phosphate buffered saline (PBS), and stored at 2-8° C. Upon initiation of the process, biopsies are inspected, and accepted biopsy tissues are washed prior to enzymatic digestion. After washing, a Liberase Digestive Enzyme Solution is added without mincing, and the biopsy tissue is incubated at 37.0 ± 2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Other commercially available collagenases may also be used, such as Serva Collagenase NB6 (Helidelburg, Germany).

After digestion, Iscove’s Complete Growth Media (IMDM, GA, 10% Fetal Bovine Serum (FBS)) is added to neutralize the enzyme, and cells are pelleted by centrifugation and re-suspended in 5.0 mL IMDM. Alternatively, full enzymatic inactivation is achieved by adding IMDM without centrifugation. Additional IMDM is added prior to seeding of the cell suspension into a particular flask (such as a T-175 cell culture flask) for initiation of cell growth and expansion. Alternatively, a T-75, T-150, T-185, or T-225 flask can be used in place of the T-175 flask. Cells are incubated at 37.0 ± 2° C. with 5.0 ± 1.0% CO₂ and supplemented with fresh IMDM every three to five days by removing half of the IMDM and replacing it with the same volume with fresh media. Alternatively, full IMDM replacements can be performed.

In specific cases, cells are not cultured in the T-175 flask for more than 30 days prior to passaging. Confluence is monitored throughout the process to ensure adequate seeding densities upon culture splitting. When cell confluence is greater than or equal to 40% in the T-175 flask, the cells are passaged by removing the spent media, washing the cells, and treating the cells with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF), or a Two Layer Cell Stack (2 CS) can be used in place of the T-500 Flask.

Morphology may be evaluated at each passage and prior to harvest to monitor culture purity throughout the process by comparing the observed sample with visual standards for morphological examination of cell cultures. Typical fibroblast morphologies when growing in cultured monolayers include elongated, fusiform, or spindle-shaped cells with slender extensions or larger, flattened stellate cells with cytoplasmic leading edges. A mixture of these morphologies may also be observed. Fibroblasts in less confluent areas can be similarly shaped but randomly oriented. The presence of keratinocytes in cell cultures may also be evaluated. Keratinocytes are round and irregularly shaped and, at higher confluence, appear to be organized in a cobblestone formation. At lower confluence, keratinocytes are observable in small colonies.

Cells are incubated at 37 ± 2° C. with 5.0 ± 1.0% CO₂ and passaged every three to five days for cells in T-500 flasks and every five to seven days for cells in ten layer cell stacks (10CS). Cells should not be cultured for more than 10 days prior to passaging. When cell confluence in a T-500 flask is ≥95%, cells are passaged to a 10 Layer Cell Stack (10 CS) culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional IMDM is added to neutralize the trypsin, and the cells from the T-500 flask are pipetted into a 2 L bottle containing fresh IMDM.

The contents of the 2 L bottle may be transferred into the 10 CS and seeded across all layers of the 10 CS. Cells are then incubated at 37.0 ± 2° C. with 5.0 ± 1.0% CO₂ and supplemented with fresh IMDM every five to seven days. In some cases, cells should not be cultured in the 10 CS for more than 20 days prior to passaging. In one embodiment, the passaged fibroblasts are rendered substantially free of immunogenic proteins present in the culture medium by incubating the expanded fibroblasts for a period of time in protein-free medium. When cell confluence in the 10 CS is ≥95%, cells are harvested. Harvesting is performed by removing the spent media, washing the cells, treating with Trypsin-EDTA to release adherent cells into the solution, and adding additional IMDM to neutralize the trypsin. Cells are collected by centrifugation and resuspended, and quality control testing is performed to determine total viable cell count and cell viability as well as sterility and the presence of endotoxins.

In some embodiments, the fibroblast dosage formulation is a suspension of fibroblasts obtained from a biopsy using standard tissue culture procedures. The cells in the formulation display typical fibroblast morphologies when growing in cultured monolayers. Specifically, cells may display an elongated, fusiform or spindle appearance with slender extensions, or cells may appear as larger, flattened stellate cells which may have cytoplasmic leading edges. A mixture of these morphologies may also be observed. The cells may also express proteins characteristic of normal fibroblasts, including the fibroblast-specific marker CD90 (Thy-1), a 35 kDa cell-surface glycoprotein, and the extracellular matrix protein collagen.

In one embodiment, regenerative fibroblast cells are purified from cord blood. Cord blood fibroblast cells are fractionated, and the fraction with enhanced therapeutic activity is administered to the patient. In some embodiments, cells with therapeutic activity are enriched based on physical differences (e.g., size and weight), electrical potential differences (e.g., charge on the membrane), differences in uptake or excretion of certain compounds (e.g., rhodamine-123 efflux), as well as differences in expression marker proteins (e.g., CD73). Distinct physical property differences between stem cells with high proliferative potential and low proliferative potential are known. Accordingly, in some embodiments, cord blood fibroblast cells with a higher proliferative ability are selected, whereas in other embodiments, a lower proliferative ability is desired. In some embodiments, cells are directly injected into the area of need and should be substantially differentiated. In other embodiments, cells are administered systemically and should be less differentiated, so as to still possess homing activity to the area of need.

In embodiments where specific cellular physical properties are the basis of differentiating between cord blood fibroblast cells with various biological activities, discrimination on the basis of physical properties can be performed using a Fluorescent Activated Cell Sorter (FACS), through manipulation of the forward scatter and side scatter settings. Other embodiments include methods of separating cells based on physical properties using filters with specific size ranges, density gradients, and pheresis techniques. In embodiments where differentiation is based on electrical properties of cells, techniques such as electrophotoluminescence are used in combination with a cell sorting means such as FACS. In some embodiments, selection of cells is based on ability to uptake certain compounds as measured by the ALDESORT system, which provides a fluorescent-based means of purifying cells with high aldehyde dehydrogenase activity. Without being bound by theory, cells with high levels of this enzyme are known to possess higher proliferative and self-renewal activities in comparison to cells possessing lower levels. Further embodiments include methods of identifying cells with high proliferative activity by identifying cells with ability to selectively efflux certain dyes such as rhodamine-123, Hoechst 33342, or a combination thereof. Without being bound to theory, cells possessing this property often express the multidrug resistance transport protein ABCG2 and are known for enhanced regenerative ability compared to cells which do not possess this efflux mechanism.

In some embodiments, cord blood cells are purified for certain therapeutic properties based on the expression of markers. In one particular embodiment, cord blood fibroblast are purified for cells with the endothelial precursor cell phenotype. Endothelial precursor cells or progenitor cells expressing markers such as but not limited to CD133, CD34, or a combination thereof and/or fibroblasts expressing markers such as but not limited to CD90, CD73, CD105 and HLA-G may be purified by positive or negative selection using techniques such as magnetic activated cell sorting (MACS), affinity columns, FACS, panning, other means known in the art, or a combination thereof. In some embodiments, cord blood-derived endothelial progenitor cells are administered directly into the target tissue, while in other embodiments, the cells are administered systemically. In some embodiments, the endothelial precursor cells are differentiated in vitro and infused into a patient. Verification of endothelial differentiation is performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

In some embodiments, cord blood fibroblast cells are endowed with desired activities prior to administration into the patient. In one specific embodiment, cord blood cells are “activated” ex vivo by brief culture in hypoxic conditions to upregulate nuclear translocation of the HIF-1α transcription factor and endow the cord blood cells with enhanced angiogenic potential. In some embodiments, hypoxia is achieved by culture of cells in conditions of 0.1% oxygen to 10% oxygen. In further embodiments, hypoxia is achieved by culture of cells in conditions of 0.5% oxygen and 5% oxygen. In further embodiments, hypoxia is achieved by culture of cells in conditions of about 1% oxygen. Cells may be cultured for a variety of time points ranging from 1 hour to 72 hours in some embodiments, to 13 hours to 59 hours in further embodiments and around 48 hours in still further embodiments. In one embodiment, cord blood cells are assessed for angiogenic or other desired activities prior to administration of the cord blood cells into the patient. Assessment methods are known in the art and include measurement of angiogenic factors, the ability to support cell viability and activity, and the ability to induce regeneration of the cellular components.

In additional embodiments, cord blood fibroblast cells are endowed with additional therapeutic properties through treatment ex vivo with factors such as de-differentiating compounds, proliferation-inducing compounds, compounds known to endow and/or enhance cord blood cells with useful properties, or a combination thereof. In one embodiment, cord blood cells are cultured with an inhibitor of the enzyme GSK-3 to enhance expansion of cells with pluripotent characteristics while maintaining the rate of differentiation. In another embodiment, cord blood cells are cultured in the presence of a DNA methyltransferase inhibitor such as 5-azacytidine to confer a “de-differentiation” effect. In another embodiment cord blood fibroblast cells are cultured in the presence of a differentiation agent that induces the cord blood stem cells to generate enhanced numbers of cells useful for treatment after the cord blood cells are administered to a patient.

In one embodiment, regenerative fibroblasts are purified from placental tissues. In contrast to cord blood fibroblast cells, in some embodiments, placental fibroblast cells are purified directly from placental tissues including the chorion, amnion, and villous stroma. In another embodiment, placental tissue is mechanically degraded in a sterile manner and treated with enzymes to allow dissociation of the cells from the extracellular matrix. Such enzymes include but are not restricted to trypsin, chymotrypsin, collagenases, elastase, hyaluronidase, or a combination thereof. In some embodiments, placental cell suspensions are subsequently washed, assessed for viability, and used directly by administration locally or systemically. In some embodiments, placental cell suspensions are purified to obtain certain populations with increased biological activity.

Purification may be performed using means known in the art including those used for purification of cord blood fibroblast cells. In some embodiments, purification may be achieved by positive selection for cell markers including SSEA3, SSEA4, TRA1-60, TRA1-81, c-kit, and Thy-1. In some embodiments, cells are expanded before introduction into the human body. Expansion can be performed by culture ex vivo with specific growth factors. Embodiments described for cord blood and embryonic stem cells also apply to placental stem cells.

Fibroblasts can also be extracted from various tissues including but not limited to umbilical cord, skin, adipose tissue, bone marrow, cord blood, and omental tissue. In some embodiments, fibroblasts are obtained from a source selected from the group comprising dermal fibroblasts; placental fibroblasts; omental tissue fibroblasts; adipose fibroblasts; bone marrow fibroblasts; foreskin fibroblasts; umbilical cord fibroblasts; cord blood fibroblasts; amniotic fluid; embryonic fibroblasts; hair follicle-derived fibroblasts; nail-derived fibroblasts; endometrial-derived fibroblasts; keloid-derived fibroblasts; ear lobe skin; plastic surgery-related by-products; or a combination thereof. In some embodiments, fibroblasts are fibroblasts isolated from skin, placenta, omentum, adipose tissue, bone marrow, foreskin, umbilical cord, cord blood, amnion, embryos, hair follicle, nails, endometrium, keloids, ear lobe, Wharton’s Jelly, and/or plastic surgery-related by-products.

In some embodiments, the fibroblasts are fibroblasts isolated from peripheral blood of a subject who has been exposed to conditions sufficient to stimulate fibroblasts from the subject to enter the peripheral blood. In another embodiment, fibroblast cells are mobilized by use of a mobilizing agent or therapy for treatment of blood vessel degeneration or aneurysms. In some embodiments, the conditions and/or agents sufficient to stimulate fibroblasts from the subject to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof. In some embodiments, the mobilization therapy is selected from a group comprising exercise, hyperbaric oxygen, autohemotherapy by ex vivo ozonation of peripheral blood, induction of SDF-1 secretion in an anatomical area outside of the bone marrow, or a combination thereof. In some embodiments, the committed fibroblasts can express the marker CD133 or CD34 and are mobilized.

Any of the fibroblast cell populations disclosed herein may be used as a source of conditioned media and/or exosomes, apoptotic bodies, or microvesicles produced by fibroblasts. The cells may be cultured alone, or may by cultured in the presence of other cells in order to further upregulate production of growth factors in the conditioned media. In some embodiments, fibroblasts of the present disclosure are cultured with an inhibitor of mRNA degradation. In some embodiments, fibroblasts are cultured under conditions suitable to support differentiation and/or reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/l, 4.5 g/l, 4 g/l, 3 g/l, 2 g/l or 1 g/l. In some embodiments, such conditions comprise glucose of about 1 g/l.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time.” Fibroblast cells used in the disclosed methods can undergo at least 25, 30, 35, or 40 doublings prior to reaching a senescent state. Methods for deriving cells capable of doubling to reach 10¹⁴ cells or more are provided. In some embodiments, methods are used to derive cells that can double sufficiently to produce at least about 10¹⁴, 10¹⁵, 10¹⁶, or 10¹⁷ or more cells when seeded at from about 10³ to about 10⁶ cells/cm² in culture within 80, 70, or 60 days or less. In some embodiments, fibroblasts are transfected with one or more genes to allow for enhanced growth and overcoming of the Hayflick limit.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick’s limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some embodiments, the present disclosure utilizes exosomes derived from fibroblasts as a therapeutic modality. Exosomes derived from fibroblasts may be used in addition to, or in place of, fibroblasts in the various methods and compositions disclosed herein. Exosomes, also referred to as “microparticles” or “particles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/ml and may float on sucrose gradients. The microparticles may be enriched in cholesterol and sphingomyelin, and lipid raft markers such as GM1, GM3, flotillin and the src protein kinase Lyn. The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the fibroblasts for the purpose of, for example, treating or preventing ovarian failure. The microparticle may comprise a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins.

As disclosed herein, fibroblasts may secrete one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts are manipulated such that they do not produce one or more factors. Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts are transfected with one or more angiogenic genes to enhance ability to promote blood vessel repair. An “angiogenic gene” describes a gene encoding for a protein or polypeptide capable of stimulating or enhancing angiogenesis in a culture system, tissue, or organism. Examples of angiogenic genes which may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of disease treatment or prevention.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-y, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

Conditions promoting certain types of fibroblast proliferation or differentiation can also be used during the culture of regenerative fibroblasts cells. These conditions include but are not limited to one or more of alteration in temperature, oxygen/carbon dioxide content, and/or turbidity of the growth media, and/or exposure to small molecule modifiers of cell culture like nutrients, certain enzyme inhibitors, certain enzyme stimulators, and/or histone deacetylase inhibitors, such as valproic acid.

In some embodiments, fibroblast cells are cultured under conditions to suppress expression of one or more apoptosis-associated genes. Anti-apoptosis genes allow for enhanced survival of fibroblasts in vitro and in vivo. In some embodiments, the one or more apoptosis-associated genes are selected from the group consisting of Fas, FasL, CASP1 (ICE), CASP10 (MCH4), CASP14, CASP2, CASP3, CASP4, CASP5, CASP6, CASP7, CASP8, CASP9, CFLAR (CASPER), CRADD, PYCARD (TMS1/ASC), ABL1, AKT1, BAD, BAK1, BAX, BCL2L11, BCLAF1, BID, BIK, BNIP3, BNIP3L, CASP1 (ICE), CASP10 (MCH4), CASP14, CASP2, CASP4, CASP6, CASP8, CD70 (TNFSF7), CIDEB, CRADD, FADD, FASLG (TNFSF6), HRK, LTA (TNFB), NOD1 (CARD4), PYCARD (TMS1/ASC), RIPK2, TNF, TNFRSF10A, TNFRSF10B (DR5), TNFRSF25 (DR3), TNFRSF9, TNFSF10 (TRAIL), TNFSF8, TP53, TP53BP2, TRADD, TRAF2, TRAF3, TRAF4, and a combination thereof.

In some embodiments, the conditions to suppress expression of apoptosis-associated genes comprise administration of an antisense oligonucleotide which activates RNAse H. In some embodiments, the conditions to suppress expression of apoptosis-associated genes comprise administration of one or more agents capable of inducing RNA interference, including short interfering RNA and/or short hairpin RNA.

In some embodiments, fibroblasts are cultured under hypoxic conditions prior to administration in order to confer enhanced cytokine production properties and stimulate migration toward chemotactic gradients. Without wishing to be bound theory, protocols to enhance the regenerative potential of non-fibroblast cells using hypoxia can be modified or adapted for use with fibroblasts. For example, in one study, short-term exposure of MSCs to 1% oxygen increased mRNA and protein expression of the chemokine receptors CX3CR1 and CXCR4. After 1-day exposure to low oxygen, in vitro migration of MSCs in response to the fractalkine and SDF-1α increased in a dose dependent manner, while blocking antibodies for the chemokine receptors significantly decreased migration. Xenotypic grafting of cells from hypoxic cultures into early chick embryos demonstrated more efficient grafting of cells from hypoxic cultures compared to cells from normoxic cultures, and cells from hypoxic cultures generated a variety of cell types in host tissues. Other descriptions of hypoxic conditioning are described in the art. For example, cells can be cultured in hypoxic conditions or with gases that displace oxygen and/or cells can be treated with hypoxic mimetics.

In some embodiments chemical agents such as iron chelators, for example, deferoxamine, are added during in vitro incubation or in vivo to enhance migration of fibroblasts to an area in need. Another useful preconditioning agent is all trans retinoic acid, used at concentrations similar to those described for MSC, for example, between 0.001 µM to 1 µM or between 0.01 µM and 1 µM.

Without wishing to be bound by theory, hypoxia has been demonstrated to induce expression of angiogenic genes in cells. For example, studies involving hypoxic preconditioning (HPC) of MSC exposed MSCs to 0.5% oxygen for 24, 48, or 72 h before evaluating the expression of pro-survival, proangiogenic, and functional markers, such as hypoxia-inducible factor-1α, VEGF, phosphorylated Akt, survivin, p21, cytochrome c, caspase-3, caspase-7, CXCR4, and c-Met. MSCs exposed to 24-h hypoxia showed reduced apoptosis and had significantly higher levels of prosurvival, proangiogenic, and pro-differentiation proteins compared to MSCs exposed to 72-h hypoxia. Cells taken directly from a cryopreserved state did not respond as effectively to 24-h HPC as those cells cultured under normoxia before HPC. Cells cultured under normoxia before HPC showed decreased apoptosis and enhanced expression of connexin-43, cardiac myosin heavy chain, and CD31. The preconditioned cells were also able to differentiate into cardiovascular lineages. The results of the study suggest that MSCs cultured under normoxia before 24-h HPC are in a state of optimal expression of prosurvival, proangiogenic, and functional proteins that may increase subsequent survival after engraftment of the cells. The same conditions used to culture MSCs can be used to culture the fibroblasts of the present disclosure.

Thus, in some embodiments, fibroblast cells are exposed to 0.1% to 10% oxygen for a period of 30 minutes to 3 days. In some embodiments, fibroblast cells are exposed to 3% oxygen for 24 hours. In some embodiments, regenerative cells are exposed to cobalt (II) chloride to chemically induce hypoxia. In some embodiments, fibroblast cells are exposed to cobalt (II) chloride for 1 to 48 hours. In some embodiments, fibroblast cells are exposed to cobalt (II) chloride for 24 hours. In some embodiments, fibroblast cells are exposed to 1 µM-300 µM cobalt (II) chloride. In some embodiments, fibroblast cells are exposed to 250 µM cobalt (II) chloride. Hypoxia can induce an upregulation in HIF-1α, which can be detected by expression of VEGF secretion. Hypoxia can also induce an upregulation of CXCR4 on fibroblast cells, which promotes homing of the cells to an SDF-1 gradient in inflamed areas.

In some embodiments, the method optionally includes enriching populations of fibroblast cells. In one embodiment, cells are transfected with a polynucleotide vector containing a stem cell-specific promoter operably linked to a reporter or selection gene. In some embodiments, the cell-specific promoter is an Oct-4, Nanog, Sox-9, GDF3, Rex- 1, or Sox-2 promoter. In some embodiments, the method further includes the step of enriching the population for the regenerative fibroblast cells using expression of a reporter or selection gene. In some embodiments, the method further includes the step of enriching the population of the regenerative fibroblast cells by flow cytometry. In a further embodiment, the method further comprises the steps of selecting fibroblast cells expressing CD 105 and/or CD 117 and transfecting the fibroblast cells expressing CD 105 and/or CD 117 with the NANOG gene.

In another embodiment, the method further includes the steps of contacting the fibroblast cells with a detectable compound that enters the cells, the compound being selectively detectable in proliferating and non-proliferating cells and enriching the population of cells for the proliferating cells. In some embodiments, the detectable compound is carboxyfluorescein diacetate, succinimidyl ester, or Aldefluor.

In some embodiments, fibroblast regenerative cells comprise fibroblast side population cells isolated based on expression of the multidrug resistance transport protein (ABCG2) or the ability to efflux intracellular dyes such as rhodamine-123 and or Hoechst 33342. Without being bound to theory, cells possessing this property express stem-like genes and are known for enhanced regenerative ability compared to cells which do not possess this efflux mechanism. Fibroblast side population cells are derived from tissues including pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer’s patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, or a combination thereof.

### B. Hematopoietic Stem Cells

Hematopoietic stem cells of the present disclosure can be administered together with fibroblasts to inhibit and/or treat aneurysms and other degenerated blood vessels. The hematopoietic stem cells may be extracted from sources known in the art such as cord blood, peripheral blood, mobilized peripheral blood, adipose tissue, and bone marrow. Additionally, hematopoietic stem cells may by generated in vitro by differentiating pluripotent stem cells or other precursor cell populations.

The hematopoietic stem cells may be autologous, allogeneic, and/or xenogenic. If allogeneic cells are used, steps to remove immunogenic components and protect against immune-mediated rejection by the patient may be taken. For example, hematopoietic stem cells may be purified substantially of contaminating leukocytes. Said purification procedures are known in the art and include selection for markers associated with hematopoietic stem cells such as CD34 and/or CD133. Steps can also include encapsulation, co-administration of an immune suppressive agent, transfection of said cells with an immune suppressory agent, or a combination thereof. Tolerance to the cells can also be induced through immunological means. Matching of allogeneic hematopoietic stem cells may be accomplished by use of HLA typing or procedures such as mixed lymphocyte reaction testing as previously described in PCT/US2007/020415.

### C. Embryonic Stem Cells

In some embodiments, blood vessel function may be restored by administration of fibroblasts together with embryonic stem cells. The embryonic stem cells can be totipotent and may express one or more antigens selected from a group consisting of: stage-specific embryonic antigens (SSEA) 3, SSEA 4, Tra-1-60 and Tra-1-81, Oct-¾, Cripto, gastrin-releasing peptide (GRP) receptor, podocalyxin-like protein (PODXL), Rex-1, GCTM-2, Nanog, and human telomerase reverse transcriptase (hTERT).

### D. Non-Embryonic Stem Cells

In some embodiments, blood vessel function may be restored by administration of fibroblasts together with non-embryonic stem cells. Non-embryonic stem cells may be derived from cord blood stem cells possessing multipotent properties and capable of differentiating into endothelial, smooth muscle, and neuronal cells. Useful cord blood stem cells may be identified based on expression of one or more antigens selected from a group consisting of SSEA-3, SSEA-4, CD9, CD34, c-kit, OCT-4, Nanog, and CXCR-4. Further, cord blood stem cells do not express one or more markers selected from a group consisting of CD3, CD34, CD45, and CD11b.

### E. Placental Stem Cells

In some embodiments, blood vessel function may be restored by administration of fibroblasts together with placental stem cells. Placental stem cells can be isolated from the placental structure and administered for regeneration of blood vessel function. Placental stem cells can be identified based on expression of one or more antigens selected from a group consisting of Oct-4, Rex-1, CD9, CD13, CD29, CD44, CD166, CD90, CD105, SH-3, SH-4, TRA-1-60, TRA-1-81, SSEA-4, and Sox-2.

### F. Bone Marrow Stem Cells

In some embodiments, blood vessel function may be restored by administration of fibroblasts together with bone marrow stem cells. Bone marrow stem cells can be isolated from the bone marrow and administered for regeneration of blood vessel function. Bone marrow stem cells may be bone marrow derived mononuclear cells, the mononuclear cells containing populations capable of differentiating into one or more cell types including endothelial cells, smooth muscle cells, neuronal cells, or a combination thereof. In some embodiments, bone marrow stem cells may be selected based on expression of one or more antigens including CD34, c-kit, flk-1, Stro-1, CD105, CD73, CD31, CD146, vascular endothelial-cadherin, CD133, CXCR-4, or a combination thereof. Additionally, stem cell activity may be enhanced by selecting for cells expressing the marker CD133.

### G. Amniotic Fluid Stem Cells

In some embodiments, blood vessel function may be restored by administration of fibroblasts together with amniotic fluid stem cells. Amniotic fluid stem cells can be isolated from amniotic fluid and administered for regeneration of blood vessel function. Amniotic fluid stem cells may be isolated by purifying mononuclear cells and/or c-kit-expressing cells from amniotic fluid, and the fluid may be extracted by means known to one of skill in the art, including utilization of ultrasound guidance. The amniotic fluid stem cells may be selected based on expression of one or more antigens including SSEA3, SSEA4, Tra-1-60, Tra-1-81, Tra-2-54, HLA class I, CD13, CD44, CD49b, CD105, Oct-4, Rex-1, DAZL, Runx-1, or a combination thereof. The amniotic fluid stem cells may also be selected based on lack of significant expression of one or more antigens including CD34, CD45, HLA Class II, or a combination thereof.

### H. Neuronal Stem Cells

In some embodiments, blood vessel function may be restored by administration of fibroblasts together with neuronal stem cells. Neuronal stem cells are selected based on expression of one or more of the following antigens: RC-2, 3CB2, BLB, Sox-2hh, GLAST, Pax 6, nestin, Muashi-1, NCAM , A2B5 and prominin.

### I. Peripheral Blood Stem Cells

In some embodiments, blood vessel function may be restored by administration of fibroblasts together with peripheral blood stem cells. Circulating peripheral blood stem cells are characterized by ability to proliferate in vitro for a period of over 3 months and by expression of CD34, CXCR4, CD117, CD113, c-met, or a combination thereof, and lack of differentiation-associated markers including CD2, CD3, CD4, CD11, CD11a, Mac-1, CD14, CD16, CD19, CD24, CD33, CD36, CD38, CD45, CD56, CD64, CD68, CD86, CD66b, HLA-DR, or a combination thereof.

### J. Mesenchymal Stem Cells

In some embodiments, blood vessel function may be restored by administration of fibroblasts together with mesenchymal stem cells. Mesenchymal stem cells express one or more markers including STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, THY-1, or a combination thereof, and do not express substantial levels of HLA-DR, CD117, CD45, or a combination thereof. Mesenchymal stem cells can be derived from bone marrow, adipose tissue, endometrium, menstrual blood, umbilical cord blood, placental tissue, peripheral blood mononuclear cells, differentiated embryonic stem cells, and differentiated progenitor cells.

### K. Germinal Stem Cells

In some embodiments, blood vessel function may be restored by administration of fibroblasts together with germinal stem cells. Germinal stem cells express markers including Oct4, Nanog, Dppa5 Rbm, cyclin A2, Tex18, Stra8, Dazl, beta1- and alpha6-integrins, Vasa, Fragilis, Nobox, c-Kit, Sca-1, Rex1, or a combination thereof.

### L. Adipose Tissue-Derived Stem Cells

In some embodiments, blood vessel function may be restored by administration of fibroblasts together with adipose tissue-derived stem cells. Adipose tissue-derived stem cells express markers including CD13, CD29, CD44, CD63, CD73, CD90, CD166, Aldehyde dehydrogenase (ALDH), ABCG2, or a combination thereof. Adipose tissue-derived stem cells can be a population of purified mononuclear cells extracted from adipose tissue capable of proliferating in culture for more than 1 month.

### M. Exfoliated Teeth-Derived Stem Cells

In some embodiments, blood vessel function may be restored by administration of fibroblasts together with exfoliated teeth-derived stem cells. Exfoliated teeth-derived stem cells express markers including STRO-1, CD146 (MUC18), alkaline phosphatase, MEPE, bFGF, or a combination thereof.

### N. Hair Follicle Stem Cells

In some embodiments, blood vessel function may be restored by administration of fibroblasts together with hair follicle stem cells. Hair follicle stem cells express markers including cytokeratin 15, Nanog, Oct-4, or a combination thereof. Hair follicle stem cells are capable of proliferating in culture for a period of at least one month. They can also secrete one or more of the following proteins when grown in culture: basic fibroblast growth factor (bFGF), endothelin-1 (ET-1), stem cell factor (SCF), or a combination thereof.

### O. Dermal Stem Cells

In some embodiments, blood vessel function may be restored by administration of fibroblasts together with dermal stem cells. Dermal stem cells express markers including CD44, CD13, CD29, CD90, CD105, or a combination thereof, and dermal stem cells are capable of proliferating in culture for a period of at least one month.

### P. Parthenogenically-Derived Stem Cells

In some embodiments, blood vessel function may be restored by administration of fibroblasts together with parthenogenically-derived stem cells. Parthenogenically-derived stem cells may be generated by adding a calcium flux-inducing agent to activate an oocyte, followed by enrichment of cells expressing markers including SSEA-4, TRA 1-60, TRA 1-81, or a combination thereof.

### Q. Reprogrammed Stem Cells

In some embodiments, blood vessel function may be restored by administration of cells which have been reprogrammed to dedifferentiate into stem cells and/or stem-like cells. Cells can be treated with a dedifferentiating agent before being used to inhibit aneurysm progression. The dedifferentiating agent can include valproic acid, lithium, and/or 5-azacytidine. Treatment with a dedifferentiating agent can induce expression of one or more markers including OCT-4, alkaline phosphatase, Sox2, TDGF-1, SSEA-3, SSEA-4, TRA-1-60, and/or TRA-1-80 [29, 30].

Dedifferentiated cells may be cultured in a multilayer population or embryoid body for a time sufficient for blood vessel cells, for example, endothelial cells, or blood vessel-like cells to develop in the culture. The time may comprise at least about 1 week, at least about 2 weeks, at least about 3 weeks, at least about 4 weeks, at least about 5 weeks, at least about 6 weeks, or at least about 7 weeks, at least about 8 weeks. The multilayer population or embryoid body may be cultured in a medium comprising DMEM. The medium may further comprise EB-DM. Dedifferentiated cells may be also be cultured on a matrix which may be selected from the group consisting of laminin, fibronectin, vitronectin, proteoglycan, entactin, collagen, collagen I, collagen IV, collagen VIII, heparan sulfate, Matrigel™ (a soluble preparation from Engelbreth-Holm-Swarm (EHS) mouse sarcoma cells), CellStart, a human basement membrane extract, and any combination thereof.

Embryoid bodies may be cultured in suspension and/or as an adherent culture (e.g., in suspension followed by adherent culture). Embryoid bodies cultured as an adherent culture may produce one or more outgrowths comprising endothelial cells. Said endothelial stem cells have reduced HLA antigen complexity.

Endothelial cells from the culture of dedifferentiated cells may be isolated and further cultured, thereby producing a population of endothelial cells useful for transplantation. Isolation may comprise dissociating cells or clumps of cells from the culture enzymatically, chemically, or physically and selecting endothelial cells.

### R. Side Population Stem Cells

In some embodiments, blood vessel function may be restored by administration of stem cells isolated as a side population of cells. Side population stem cells can be isolated based on expression of the multidrug resistance transport protein (ABCG2) or the ability to efflux intracellular dyes such as rhodamine-123 and or Hoechst 33342. Without being bound to theory, cells possessing this property express stem-like genes and are known for enhanced regenerative ability compared to cells which do not possess this efflux mechanism Side population stem cells can be derived from tissues including pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer’s patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, or a combination thereof.

### IV. Generation of Fibroblast-Conditioned Media

The present disclosure provides means of inhibiting and/or treating aneurysms and other degenerated blood vessels through administration of a fibroblast cell population and/or fibroblast-derived products or fibroblast-produced therapeutic factors. Fibroblast-produced therapeutic factors can include growth media conditioned by fibroblasts, for example. In some embodiments, fibroblast cells are cultured in a growth medium to obtain conditioned media. In some embodiments, fibroblasts are cultured directly in tissue culture media including DMEM, EMEM, IMEM, or RPMI to produce fibroblast-conditioned media. In some embodiments, fibroblast-conditioned media is generated by culturing fibroblasts in hypoxic and/or hyperthermic conditions and/or with histone deacetylase inhibitors. In some embodiments, fibroblasts are also cultured alone or cultured in the presence of other cells to further upregulate production of growth factors in the conditioned media. Methods for generating conditioned media from fibroblasts are described herein.

Conditioned medium may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500 x g). Conditioned media may be filtered through a membrane. The membrane may be a >1000 kDa membrane. Conditioned media may be subject to liquid chromatography such as HPLC. Conditioned media may be separated by size exclusion.

Fibroblasts may be expanded and utilized by administration themselves, or may be cultured in a growth media in order to obtain conditioned media. The term Growth Medium generally refers to a medium sufficient for the culturing of fibroblasts. In particular, one presently preferred medium for the culturing of the cells herein comprises Dulbecco’s Modified Essential Media (DMEM). Particularly preferred is DMEM-low glucose (also DMEM-LG herein) (Invitrogen®, Carlsbad, Calif.). The DMEM-low glucose is preferably supplemented with 15% (v/v) fetal bovine serum (e.g. defined fetal bovine serum, HycloneTM, Logan Utah), antibiotics/antimycotics (preferably penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases different growth media are used, or different supplementations are provided, and these are normally indicated as supplementations to Growth Medium. Also relating to the present disclosure, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

In one embodiment, the conditioned media comprises a liquid which has been in contact with fibroblast cells. In some embodiments, conditioned media is generated by culturing fibroblasts. In some embodiments, conditioned media is generated by combining fibroblasts with immune cells in a liquid media. Fibroblast cells may be fibroblasts that grow in an undifferentiated state and/or may be fibroblasts which have been stimulated with an agent that mimics inflammation and/or danger signals. Such agents are administered to fibroblasts in order to upregulate production of cytokines that are useful for the treatment of blood vessel degeneration. In some embodiments cytokines secreted by fibroblasts whose upregulation is stimulated by exposure to inflammatory-related signals includes one or more of MCP-1, IL-6, IL-8, GCP-2, HGF, KGF, FGF, HB-EGF, BDNF, TPO, MIP1a, RANTES, TIMP1, HMGB1, IL-18, IL-33, or a combination thereof. In some embodiments fibroblasts are stimulated with agonists of toll-like receptors, including one or more of Triacyl lipopeptides, lipopeptides, lipoteichoic acid, HSP70, zymosan (β-glucan), double-stranded RNA, poly I:C, lipopolysaccharide, fibrinogen, heparan sulfate fragments, hyaluronic acid fragments, Bacterial flagellin, multiple diacyl lipopeptides, imidazoquinoline, loxoribine (a guanosine analogue), bropirimine, resiquimod, unmethylated CpG Oligodeoxynucleotide DNA, triacylated lipopeptides, Profilin, bacterial ribosomal RNA sequence “CGGAAAGACC,” or a combination thereof.

In one embodiment, provided is a means of creating a medicament useful for the treatment of blood vessel degeneration by culturing cells in a serum free media. Many types of media may be chosen and used by one of skill in the art. In one embodiment, a media is selected from the group consisting of alpha MEM, DMEM, RPMI, Opti-MEM, IMEM, and AIM-V Cells may be cultured in a variety of expansion media that contain fetal calf serum or other growth factors. However, for collection of therapeutic supernatant, in a specific embodiment, the cells are transferred to a media substantially lacking serum. In some embodiments, the supernatant is administered directly into the patient in need of treatment. It is well known in the art that preparation of the supernatant before administration may be performed by various means; for example, the supernatant may be filter sterilized or in some conditions concentrated. In a specific embodiment, the supernatant is administrated intramuscularly, orally, sublingually, intranasally, intraventricularly, intrarectally, intravenously, and/or intrathecally.

In some embodiments, culture conditioned media is concentrated by filtering/desalting means known in the art. In one embodiment, filters with specific molecular weight cut-offs are utilized. In one embodiments, the filters select for molecular weights between 1 kDa and 50 kDa. In one embodiment, the cell culture supernatant is concentrated using means known in the art such as solid phase extraction using C18 cartridges (Mini-Speed C18-14%, S.P.E. Limited, Concord ON). C18 cartridges are used to adsorb small hydrophobic molecules from the stem or progenitor cell culture supernatant, and allows for the elimination of salts and other polar contaminants. The cartridges are prepared by washing with methanol, followed by washing with deionized-distilled water. In some embodiments, up to 100 ml of stem cell or progenitor cell supernatant may be passed through each of these specific cartridges before elution, though one of skill in the art would understand that larger cartridges may be used. After washing the cartridges, adsorbed material is eluted with methanol, evaporated under a stream of nitrogen, re-dissolved in a small volume of methanol, and stored at 4° C. Before testing the eluate for activity in vitro, the methanol is evaporated under nitrogen and replaced by culture medium. In other embodiments, different adsorption means known in the art are used to purify certain compounds from fibroblast cell supernatants.

In some embodiments, further purification and concentration is performed using gel filtration with a Bio-Gel P-2 column having a nominal exclusion limit of 1800 Da (Bio-Rad, Richmond Calif.). The column is washed and pre-swelled in 20 mM Tris-HCl buffer, pH 7.2, (Sigma) and degassed by gentle swirling under vacuum. Bio-Gel P-2 material is packed into a 1.5x.54 cm glass column and equilibrated with 3 column volumes of the same buffer. Cell supernatant concentrates extracted by filtration are dissolved in 0.5 ml of 20 mM Tris buffer, pH 7.2, and run through the column. Fractions are collected from the column and analyzed for biological activity. In alternative embodiments, other purification, fractionation, and identification means known to one skilled in the art including anionic exchange chromatography, gas chromatography, high performance liquid chromatography, nuclear magnetic resonance, and mass spectrometry are used to prepare concentrated supernatants.

In some embodiments, active supernatant fractions are administered locally or systemically. The supernatant concentrated from fibroblast-conditioned media is assessed directly for biological activities or further purified. In vitro bioassays allow for identification of the molecular weight fraction of the supernatant possessing biological activity and quantification of biological activity within the identified fractions. Production of various proteins and biomarkers associated is assessed by analysis of protein content using techniques including mass spectrometry, column chromatography, immune based assays such as enzyme linked immunosorbent assay (ELISA), immunohistochemistry, and flow cytometry.

### V. Exosomes Purified From Fibroblasts

The present disclosure provides means of inhibiting and/or treating aneurysms and other degenerated blood vessels through administration of a fibroblast cell population and/or fibroblast-produced therapeutic factors. Fibroblast-produced therapeutic factors can include exosomes, apoptotic bodies, and/or microvesicles produced by fibroblasts, for example.

In some embodiments, exosomes purified from transfected fibroblasts are used therapeutically. In some embodiments, purified fibroblast exosomes are used to decrease IL-17 production. In some embodiments, fibroblast-derived exosomes are used to suppress inflammation, including but not limited to suppressing production of IL-1, IL-6, and TNF-alpha by macrophages. In some embodiments, fibroblast-derived exosomes stimulate production of growth factors which in some cases prevent apoptosis of endothelial cells and further degeneration of blood vessels. Methods for purifying exosomes are known in the art and described herein.

In one embodiment, fibroblasts are cultured using means known in the art for preserving the viability and proliferative ability of fibroblasts. Both individualized autologous exosome preparations and exosome preparations obtained from established cell lines for experimental or biological use may be used. In one embodiment, chromatography separation methods are used to prepare membrane vesicles, particularly to separate the membrane vesicles from potential biological contaminants, wherein the membrane vesicles are exosomes and cells used to generate the exosomes are fibroblast cells.

In one embodiment, strong or weak anion exchange is performed. In addition, in a specific embodiment, the chromatography is performed under pressure. Thus, in some embodiments, the chromatography consists of high performance liquid chromatography (HPLC). Different types of column supports may be used to perform the anion exchange chromatography. In some embodiments, the column supports include cellulose, poly(styrene-divinylbenzene), agarose, dextran, acrylamide, silica, ethylene glycol-methacrylate co-polymer, or mixtures thereof, e.g., agarose-dextran mixtures. Column supports include but are not limited to gels including: SOURCE™, POROS™, SEPHAROSE™, SEPHADEX™, TRISACRYL™, TSK-GEL SW™ or PW™, SUPERDEX™, TOYOPEARL HW™, and SEPHACRYL™. Therefore, in a specific embodiment, membrane vesicles, particularly exosomes, are prepared from a biological sample such as a tissue culture containing fibroblasts, comprising at least one step during which the biological sample is purified by anion exchange chromatography on an optionally-functionalized column support selected from one or more of cellulose, poly(styrenedivinylbenzene), silica, acrylamide, agarose, dextran, ethylene glycol-methacrylate co-polymer, alone or in combinations thereof.

In some embodiments, the column supports are in bead form to improve chromatographic resolution. The beads can be homogeneous and calibrated in diameter with a sufficiently high porosity to enable the penetration of objects like exosomes undergoing chromatography. The diameter of exosomes is generally between 50 and 100 nm. Thus, in some embodiments, high porosity gels with diameters between about 10 nm and about 5 µm, about 20 nm and about 2 µm, or about 100 nm and about 1 µm are used. For anion exchange chromatography, the column support used can be functionalized with a group capable of interacting with an anionic molecule. Generally, this group is composed of a ternary or quaternary amine, which defines a weak or strong anion exchanger, respectively. In some embodiments, a strong anion exchanger corresponding to a chromatography column support functionalized with quaternary amines is used. Therefore, according to a more specific embodiment, anion exchange chromatography is performed on a column support functionalized with a quaternary amine and selected from one or more of poly(styrene-divinylbenzene), acrylamide, agarose, dextran, and silica, alone or in combinations thereof, and functionalised with a quaternary amine. Column supports functionalized with a quaternary amine include but are not limited to gels including SOURCE™ Q, MONO Q™, Q SEPHAROSE™, POROS™ HQ and POROS™ QE, FRACTOGEL™ TMAE type gels and TOYOPEARL SUPER™ Q gels.

In one embodiment, the column support used to perform the anion exchange chromatography comprises poly(styrene-divinylbenzene), for example, SOURCE Q gels like SOURCE™ 15Q (Pharmacia). This column support comprises large internal pores, low resistance to liquid circulation through the gel, and rapid diffusion of exosomes to the functional groups. Biological materials including exosomes retained on the column support may be eluted using methods known in the art, for example, by passing a saline solution gradient of increasing concentration over the column support. In some embodiments, a sodium chloride solution is used in concentrations varying from 0 to 2 M, for example. Purified fractions are detected based on optical densities (OD) of the fractions measured at the column support outlet using a continuous spectrophotometric reading. In some embodiments, fractions comprising membrane vesicles are eluted at an ionic strength of approximately 350 to 700 mM, depending on vesicle type.

Different types of chromatographic columns may be used depending on experimental requirements and volumes to be purified. For example, depending on the preparations, column volumes can vary from 100 µl up to ≥10 ml, and column supports can bind and retain up to 25 mg of proteins/ml. As an example, a 100 µl column has a capacity of approximately 2.5 mg of protein, which allows for purification of approximately 2 liters of culture supernatants concentrated by a factor of 10 to 20 to yield volumes of 100 to 200 ml per preparation. Higher volumes may also be purified by increasing the column volume.

Membrane vesicles can also be purified using gel permeation liquid chromatography. In some embodiments, the anion exchange chromatography step is combined with a gel permeation chromatography step either before or after the anion exchange chromatography step. In some embodiments, the permeation chromatography step takes place after the anion exchange step. In some embodiments, the anion exchange chromatography step is replaced with the gel permeation chromatography step. To perform gel permeation chromatography, a support selected from one or more of silica, acrylamide, agarose, dextran, ethylene glycol-methacrylate co-polymer, alone or in combinations thereof, e.g., agarose-dextran mixtures. Column supports include but are not limited to gels including SUPERDEX™ 200HR (Pharmacia), TSK G6000 (TosoHaas), and SEPHACRYL™ S (Pharmacia).

Gel permeation chromatography may be applied to different biological samples. In some embodiments, biological samples include but are not limited to biological fluid from a subject (bone marrow, peripheral blood, etc.), cell culture supernatant, cell lysate, pre-purified solution, or any other composition comprising membrane vesicles. In one embodiment, the biological sample is a culture supernatant of membrane vesicle-producing fibroblast cells treated, prior to chromatography, so as to enrich the supernatant for membrane vesicles. Thus, one embodiment relates to a method of preparing membrane vesicles from a biological sample, the method characterized by at least a) an enrichment step to prepare a sample enriched with membrane vesicles and b) a purification step during which the sample is purified by anion exchange chromatography and/or gel permeation chromatography.

In some embodiments, the biological sample is composed of an enriched, pre-purified solution obtained by centrifugation, clarification, ultrafiltration, nanofiltration and/or affinity chromatography of a cell culture supernatant of a membrane vesicle-producing fibroblast cell population or biological fluid. Thus, one embodiment relates to a method of preparing membrane vesicles comprising at least the steps of a) culturing a population of membrane vesicle-producing cells under conditions enabling the release of vesicles; b) enriching membrane vesicles in the sample; and c) performing anion exchange chromatography and/or gel permeation chromatography to purify the sample.

In some embodiments, the sample (e.g. supernatant) enrichment step comprises one or more centrifugation, clarification, ultrafiltration, nanofiltration, affinity chromatography, or a combination thereof. In one embodiment, the enrichment step comprises the steps of (i) elimination of cells and/or cell debris (clarification) and (ii) concentration and/or affinity chromatography. In one embodiment, affinity chromatography following clarification is optional. In one embodiment, the enrichment step comprises the steps of (i) elimination of cells and/or cell debris (clarification); (ii) concentration; and (iii) an affinity chromatography.

In some embodiments, the elimination step of enrichment is achieved by centrifugation of the sample, for example, at a speeds below 1000 g, such as between 100 and 700 g. In one embodiment, centrifugation conditions are approximately 300 g or 600 g for a period between 1 and 15 minutes.

In some embodiments, the elimination step of enrichment is achieved by filtration of the sample. In some embodiments, sample filtration is combined with centrifugation as described. The filtration may be performed with successive filtrations using filters with a decreasing porosity. In one embodiment, filters with a porosity between 0.2 and 10 µm are used. In one embodiment, a succession of filters with a porosities of 10 µm, 1 µm, 0.5 µm, and 0.22 µm are used.

In some embodiments, the concentration step of enrichment is performed to reduce the volume of sample to be purified during chromatography. In some embodiments, the concentration step of enrichment is achieved by centrifugation of the sample at speeds between 10,000 and 100,000 g to cause the sedimentation of the membrane vesicles. In some embodiments, the concentration step of enrichment is performed as a series of differential centrifugations, with the last centrifugation performed at approximately 70,000 g. After centrifugation, the pelleted membrane may be resuspended in a smaller volume of suitable buffer.

In some embodiments, the concentration step of enrichment is achieved by ultrafiltration which allows both to concentration of the supernatant and initial purification of the vesicles. In one embodiment, the biological sample (e.g., the supernatant) is subjected to tangential ultrafiltration consisting of concentration and fractionation of the sample between two compartments (filtrate and retentate) separated by membranes of determined cut-off thresholds. Separation of the sample is carried out by applying a flow in the retentate compartment and a transmembrane pressure between the retentate compartment and the filtrate compartment. Different systems may be used to perform the ultrafiltration, such as spiral membranes (Millipore, Amicon), flat membranes, or hollow fibers (Amicon, Millipore, Sartorius, Pall, GF, Sepracor). In some embodiments, membranes with cut-off thresholds below 1000 kDa, 300 kDa to 1000 kDa, or 300 kDa to 500 kDa are used.

The affinity chromatography step can be performed in various ways, using different chromatographic support and material known in the art. In some embodiments, nonspecific affinity chromatography aimed at retaining (i.e., binding) certain contaminants present within the solution without retaining the objects of interest (i.e., the exosomes) is used as a form of negative selection. In some embodiments, affinity chromatography on a dye is used, allowing for the elimination (i.e., the retention) of contaminants such as proteins and enzymes like albumin, kinases, deshydrogenases, clotting factors, interferons, lipoproteins, or also co-factors, etc. The supports used for affinity chromatography on a dye are the supports used for ion exchange chromatography functionalized with a dye. In some embodiments, the dye is selected from the group consisting of Blue SEPHAROSE™ (Pharmacia), YELLOW 86, GREEN 5, and BROWN 10 (Sigma). In some embodiments, the support is agarose. However, those of skill in the art will understand that any other support and/or dye or reactive group allowing the retention (binding) of contaminants from the biological sample to be purified can be used.

Thus, one embodiment relates to a method of preparing membrane vesicles comprising the steps of a) culturing a population of membrane vesicle-producing cells under conditions enabling release of the vesicles; b) treating the culture supernatant with at least one ultrafiltration or affinity chromatography step to produce a biological sample enriched with membrane vesicles; and c) using anion exchange chromatography and/or gel permeation chromatography to purify the biological sample. In some embodiments, step b) comprises filtration of the culture supernatant, followed by an ultrafiltration, including tangential ultrafiltration. In further embodiments, step b) comprises clarification of the culture supernatant, followed by an affinity chromatography on dye, including Blue SEPHAROSE™.

In some embodiments, after step c), the harvested material is subjected to d) one or more additional treatment and/or filtration steps for sterilization purposes. For step d), filters with a diameter ≤0.3 µm or ≤0.25 µm are used. In some embodiments, after step d), the sterilized, purified material obtained is distributed into suitable containers such as bottles, tubes, bags, syringes, etc., in a suitable storage medium and stored cold, frozen, or used extemporaneously. Thus, in some embodiments, the method of preparing membrane vesicles further comprises c) anion exchange chromatography purification of the biological sample and d) a sterilizing filtration step of the material harvested in step c). In further embodiments, the method of preparing membrane vesicles further comprises c) gel permeation chromatography purification of the biological sample and d) a sterilizing filtration step of the material harvested in step c). In additional embodiments, the method of preparing membrane vesicles further comprises c) anionic exchange purification of the biological sample followed or preceded by gel permeation chromatography and d) a sterilizing filtration step of the material harvested in step c).

### VI. Administration of Therapeutic Compositions

The therapy provided herein may comprise administration of a composition comprising therapeutic agents (e.g., fibroblasts, stem cells, fibroblast-produced therapeutic factors, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., fibroblasts) of the disclosure may be administered by the same route of administration or by different routes of administration. In some embodiments, the therapy is administered intravenously, intramuscularly, subcutaneously, topically, orally, sublingually, transdermally, intraperitoneally, intraorbitally, by implantation, by inhalation, intrathecally, intraventricularly, and/or intranasally. The appropriate dosage may be determined based on the type of disease to be treated, severity and course of the disease, the clinical condition of the individual, the individual’s clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, subjects are treated with fibroblasts within a composition. Fibroblasts can be activated prior to therapeutic use and/or fibroblasts can be administered with agents which act as “regenerative adjuvants” for the fibroblasts. In some embodiments, compositions containing fibroblasts comprise between about 10,000-10 million cells per kilogram of body weight. In some embodiments, approximately 1 million cells per kilogram of body weight are administered. In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹¹ cells are infused per 100 kg. In some embodiments, 100 kg of body weight is measured as 100 kg of body weight of a human.. In some embodiments, between about 50 million and about 500 million fibroblast cells are administered to the subject. For example, between about 50 million and about 100 million fibroblast cells, between about 50 million and about 200 million fibroblast cells, between about 50 million and about 300 million fibroblast cells, between about 50 million and about 400 million fibroblast cells, between about 100 million and about 200 million fibroblast cells, between about 100 million and about 300 million fibroblast cells, between about 100 million and about 400 million fibroblast cells, between about 100 million and about 500 million fibroblast cells, between about 200 million and about 300 million fibroblast cells, between about 200 million and about 400 million fibroblast cells, between about 200 million and about 500 million fibroblast cells, between about 300 million and about 400 million fibroblast cells, between about 300 million and about 500 million fibroblast cells, between about 400 million and about 500 million fibroblast cells, about 50 million fibroblast cells, about 100 million fibroblast cells, about 150 million fibroblast cells, about 200 million fibroblast cells, about 250 million fibroblast cells, about 300 million fibroblast cells, about 350 million fibroblast cells, about 400 million fibroblast cells, about 450 million fibroblast cells, or about 500 million fibroblast cells may be administered to the subject.

In some embodiments, a single administration of cells is provided. The cells can be fibroblasts and/or other cells of the disclosure. In some embodiments, cells are administered once monthly, weekly, and/or daily. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days. In some embodiments, up to about 20 administrations of between about 10⁵ and about 10¹³ cells per 100 kg are provided.

The quantity to be administered, both according to number of treatments and unit dose, depends on the treatment effect desired. An effective dose is understood to refer to an amount necessary to achieve a particular effect. In some embodiments, it is contemplated that doses between about 10⁵ and about 10¹³ cells per 100 kg are administered to affect the protective capability of the fibroblasts. In certain embodiments, it is contemplated that a cell number in the range from 10 mg/kg to 200 mg/kg can affect the protective capability of these agents. Thus, it is contemplated that doses include doses of cell numbers of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 µg/kg, mg/kg, µg/day, or mg/day or any range derivable therein. Furthermore, such doses can be administered at multiple times during a day, and/or on multiple days, weeks, or months.

In some embodiments, it is contemplated that a dose of a drug or other therapeutic agent in the range from 10 mg/kg to 200 mg/kg can affect the protective capability of these agents. Thus, it is contemplated that doses include doses of drugs or other therapeutic agents of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 µg/kg, mg/kg, µg/day, or mg/day or any range derivable therein. Furthermore, such doses can be administered at multiple times during a day, and/or on multiple days, weeks, or months.

It will be understood by those skilled in the art and made aware that dosage units of µg/kg or mg/kg of body weight can be converted and expressed in comparable concentration units of µg/ml or mM (blood levels), such as 4 µM to 100 µM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain embodiments, the effective dose of a pharmaceutical composition is one which can provide a blood level of about 1 µM to 150 µM. In another embodiment, the effective dose provides a blood level of about 4 µM to 100 µM.; or about 1 µM to 100 µM; or about 1 µM to 50 µM; or about 1 µM to 40 µM; or about 1 µM to 30 µM; or about 1 µM to 20 µM; or about 1 µM to 10 µM; or about 10 µM to 150 µM; or about 10 µM to 100 µM; or about 10 µM to 50 µM; or about 25 µM to 150 µM; or about 25 µM to 100 µM; or about 25 µM to 50 µM; or about 50 µM to 150 µM; or about 50 µM to 100 µM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 µM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### VII. Kits of the Disclosure

Any of the cellular and/or non-cellular compositions described herein or similar thereto may be comprised in a kit. In a non-limiting example, one or more reagents for use in methods for preparing fibroblasts or derivatives thereof may be comprised in a kit. Such reagents may include cells, vectors, one or more growth factors, vector(s), one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cd73-selected Fibroblasts Suppress Development of Aortic Aneurysm In An Elastase Model

For induction of aneurysms, a murine elastase perfusion model of abdominal aortic aneurysm formation was used as previously described (Sharma et al., Experimental abdominal aortic aneurysm formation is mediated by IL-17 and attenuated by mesenchymal stem cell treatment. Circulation. 2012;126:S38-45). Briefly, the infrarenal abdominal aorta was isolated in situ and perfused with porcine pancreatic elastase (Sigma, 0.4 U/mL) for 5 minutes at a pressure of 100 mm Hg. Control animals were perfused with heat-inactivated elastase for 5 minutes. Video micrometric measurements of aortic diameters were made in situ before perfusion, after perfusion, and before harvesting the aorta on days 0, 7, 14 and 21.

Human foreskin fibroblasts were obtained from American Type Culture Collection (ATCC) and grown according to the manufacturer’s instructions. Separation of the fibroblasts into CD73+ and CD73- cells was performed using magnetic activated cell sorting (MACS) according to the manufacturer’s instructions. On day 1 after elastase administration, cells were administered intravenously via the tail vein at a concentration of 500,000 cells per mouse.

As seen in FIG. 1, animals receiving saline control had a progressive increase in aortic diameter, while animals receiving CD73-negative fibroblasts possessed a decrease in expansion of aneurysm, and CD73-positive fibroblasts possessed the highest amount of aneurysm inhibitory activity.

### Example 2

### Reduction of Aneurysm by Administration of Cd73 Selected Fibroblast Derived Exosomes

Fibroblasts were cultured under conditions sufficient to promote release of exosomes into the media. Specifically, foreskin fibroblasts were cultured in 15 ml alpha MEM-media containing 10% fetal calf serum in T 175 flasks. After a 24 hour culture, media was replaced with phosphate buffered saline and cells were cultured for an additional 12 hours. Exosomes were extracted by gradient centrifugation, specifically 10 min at 300 g, 10 min at 2000 g, 30 min at 10000 g, followed by exosome pelleting by centrifugation at 100000 g for 70 min. A repeated 100000 g centrifugation of the re-suspended pellet was applied to purify the exosome preparation from the lower mobility fractions, mainly from free proteins.

For induction of aneurysm a mouse elastase model was used. The intrarenal abdominal aorta was isolated in situ and perfused with porcine elastase for 5 minutes at 0.4 U/ml at a pressure of 100 mm Hg.

Animals (10 per group) were treated with control surgery, elastase, elastase and exosomes from CD73-negative fibroblasts, and elastase and exosomes from CD73-positive fibroblasts. FIG. 2 shows prevention of the aortic aneurysm increase with the fibroblast exosomes.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

1. Sagripanti, A. and A. Carpi, Antithrombotic and prothrombotic activities of the vascular endothelium. Biomed Pharmacother, 2000. 54(2): p. 107-11.

2. Correia, M.L. and W.G. Haynes, Arterial compliance and endothelial function. Curr Diab Rep, 2007. 7(4): p. 269-75.

3. Stefano, G.B., et al., Interleukin-10 stimulation of endogenous nitric oxide release from human saphenous veins diminishes immunocyte adherence. J Cardiovasc Pharmacol, 1997. 30(1): p. 90-5.

4. Hristov, M., et al., Regulation of endothelial progenitor cell homing after arterial injury. Thromb Haemost, 2007. 98(2): p. 274-7.

5. Regnault, V. and B. Levy, Recombinant activated protein C in sepsis: endothelium protection or endothelium therapy? Crit Care, 2007. 11(1): p. 103.

6. Yavuz, B.B., et al., Advanced Age Is Associated with Endothelial Dysfunction in Healthy Elderly Subjects. Gerontology, 2008.

7. Lauer, T., et al., Age-dependent endothelial dysfunction is associated with failure to increase plasma nitrite in response to exercise. Basic Res Cardiol, 2008. 103(3): p. 291-7.

8. Hurme, M., et al., CRP gene is involved in the regulation of human longevity: a follow-up study in Finnish nonagenarians. Mech Ageing Dev, 2007. 128(10): p. 574-6.

9. Ferri, C., et al., C-reactive protein: interaction with the vascular endothelium and possible role in human atherosclerosis. Curr Pharm Des, 2007. 13(16): p. 1631-45.

10. Teoh, H., et al., Impaired endothelial function in C-reactive protein overexpressing mice. Atherosclerosis, 2008.

11. Hristov, M., et al., Importance of CXC chemokine receptor 2 in the homing of human peripheral blood endothelial progenitor cells to sites of arterial injury. Circ Res, 2007. 100(4): p. 590-7.

12. Kondo, S., et al., Apoptosis of medial smooth muscle cells in the development of saccular cerebral aneurysms in rats. Stroke, 1998. 29(1): p. 181-8; discussion 189.

13. Sangiorgi, G., et al., Plasma levels of metalloproteinases-3 and -9 as markers of successful abdominal aortic aneurysm exclusion after endovascular graft treatment. Circulation, 2001. 104(12 Suppl 1): p. I288-95.

14. Thompson, R.W. and B.T. Baxter, MMP inhibition in abdominal aortic aneurysms. Rationale for a prospective randomized clinical trial. Ann N Y Acad Sci, 1999. 878: p. 159-78.

15. Yu, X.F. and Z.C. Han, Matrix metalloproteinases in bone marrow: roles of gelatinases in physiological hematopoiesis and hematopoietic malignancies. Histol Histopathol, 2006. 21(5): p. 519-31.

16. Rao, Q., et al., Production of matrix metalloproteinase-9 by cord blood CD34+ cells and its role in migration. Ann Hematol, 2004. 83(7): p. 409-13.

17. Ghajar, C.M., et al., Mesenchymal stem cells enhance angiogenesis in mechanically viable prevascularized tissues via early matrix metalloproteinase upregulation. Tissue Eng, 2006. 12(10): p. 2875-88.

18. Gibbs, D.A., et al., A clinical trial of fibroblast transplantation for the treatment of mucopolysaccharidoses. J Inherit Metab Dis, 1983. 6(2): p. 62-81.

19. Akle, C., et al., Transplantation of amniotic epithelial membranes in patients with mucopolysaccharidoses. Exp Clin Immunogenet, 1985. 2(1): p. 43-8.

20. Wetzels, A.M., et al., The effects of human skin fibroblast monolayers on human sperm motility and mouse zygote development. Hum Reprod, 1992. 7(6): p. 852-6.

21. Hansbrough, J.F., C. Dore, and W.B. Hansbrough, Clinical trials of a living dermal tissue replacement placed beneath meshed, split-thickness skin grafts on excised burn wounds. J Burn Care Rehabil, 1992. 13(5): p. 519-29.

22. Sabolinski, M.L., et al., Cultured skin as a ‘smart material’ for healing wounds: experience in venous ulcers. Biomaterials, 1996. 17(3): p. 311-20.

23. Eaglstein, W.H., et al., Acute excisional wounds treated with a tissue-engineered skin (Apligraf). Dermatol Surg, 1999. 25(3): p. 195-201.

24. Noordenbos, J., C. Dore, and J.F. Hansbrough, Safety and efficacy of TransCyte for the treatment of partial-thickness burns. J Burn Care Rehabil, 1999. 20(4): p. 275-81.

25. Chang, D.W., et al., Can a tissue-engineered skin graft improve healing of lower extremity foot wounds after revascularization? Ann Vasc Surg, 2000. 14(1): p. 44-9.

26. McGuire, M.K., et al., A pilot study to evaluate a tissue-engineered bilayered cell therapy as an alternative to tissue from the palate. J Periodontol, 2008. 79(10): p. 1847-56.

27. Kirsner, R.S., et al., Spray-applied cell therapy with human allogeneic fibroblasts and keratinocytes for the treatment of chronic venous leg ulcers: a phase 2, multicentre, double-blind, randomised, placebo-controlled trial. Lancet, 2012. 380(9846): p. 977-85.

28. Lantis, J.C., 2nd, et al., The influence of patient and wound variables on healing of venous leg ulcers in a randomized controlled trial of growth-arrested allogeneic keratinocytes and fibroblasts. J Vasc Surg, 2013. 58(2): p. 433-9.

29. Huang, Y., et al., Histone deacetylase inhibitor significantly improved the cloning efficiency of porcine somatic cell nuclear transfer embryos. Cell Reprogram, 2011. 13(6): p. 513-20.

30. Marquez-Curtis, L.A., et al., Migration, proliferation, and differentiation of cord blood mesenchymal stromal cells treated with histone deacetylase inhibitor valproic Acid. Stem Cells Int, 2014. 2014: p. 610495.

31. Voitenok, N.N., et al., Induction of tumor necrosis factor synthesis in human monocytes treated by transcriptional inhibitors. Immunol Lett, 1989. 20(1): p. 77-82.

32. Evans, G.F. and S.H. Zuckerman, Pharmacologic modulation of TNF production by endotoxin stimulated macrophages: in vitro and in vivo effects of auranofin and other chrysotherapeutic compounds. Agents Actions, 1989. 26(3-4): p. 329-34.

33. Schade, U.F., et al., Lipoxygenase inhibitors suppress formation of tumor necrosis factor in vitro and in vivo. Biochem Biophys Res Commun, 1989. 159(2): p. 748-54.

34. Schade, U.F., R. Engel, and D. Jakobs, The role of lipoxygenases in endotoxin-induced cytokine production. Prog Clin Biol Res, 1991. 367: p. 73-82.

35. Schade, F.U., R. Engel, and D. Jakobs, Lipoxygenase inhibitors but not site specific 5-lipoxygenase blockers protect against endotoxic shock and inhibit production of tumor necrosis factor. Eicosanoids, 1992. 5 Suppl: p. S45-7.

36. Elekes, E., D. Jakobs, and F.U. Schade, Suppression of endotoxin mitogenicity of spleen cells by lipoxygenase inhibitors and its reversal by 13-hydroxyoctadecadienoic acid. FEMS Immunol Med Microbiol, 1993. 6(1): p. 13-20.

37. D′Souza, N.B., et al., Acute alcohol infusion suppresses endotoxin-induced serum tumor necrosis factor. Alcohol Clin Exp Res, 1989. 13(2): p. 295-8.

38. Bermudez, L.E., et al., Ethanol affects release of TNF and GM-CSF and membrane expression of TNF receptors by human macrophages. Lymphokine Cytokine Res, 1991. 10(5): p. 413-9.

39. Dubois, C.M., E. Bissonnette, and M. Rola-Pleszczynski, Asbestos fibers and silica particles stimulate rat alveolar macrophages to release tumor necrosis factor. Autoregulatory role of leukotriene B4. Am Rev Respir Dis, 1989. 139(5): p. 1257-64.

40. Essner, R., et al., IL-4 down-regulates IL-1 and TNF gene expression in human monocytes. J Immunol, 1989. 142(11): p. 3857-61.

41. Di Santo, E., et al., IL-13 inhibits TNF production but potentiates that of IL-6 in vivo and ex vivo in mice. J Immunol, 1997. 159(1): p. 379-82.

42. Stokes, D.C., et al., Polymyxin B prevents lipopolysaccharide-induced release of tumor necrosis factor-alpha from alveolar macrophages. J Infect Dis, 1989. 160(1): p. 52-7.

43. Pappo, I., et al., Polymyxin B reduces total parenteral nutrition-associated hepatic steatosis by its antibacterial activity and by blocking deleterious effects of lipopolysaccharide. JPEN J Parenter Enteral Nutr, 1992. 16(6): p. 529-32.

44. Greve, J.W., D.J. Gouma, and W.A. Buurman, Bile acids inhibit endotoxin-induced release of tumor necrosis factor by monocytes: an in vitro study. Hepatology, 1989. 10(4): p. 454-8.

45. Aderka, D., J.M. Le, and J. Vilcek, IL-6 inhibits lipopolysaccharide-induced tumor necrosis factor production in cultured human monocytes, U937 cells, and in mice. J Immunol, 1989. 143(11): p. 3517-23.

46. Greve, J.W., et al., Lactulose inhibits endotoxin induced tumour necrosis factor production by monocytes. An in vitro study. Gut, 1990. 31(2): p. 198-203.

47. Waage, A., M. Sorensen, and B. Stordal, Differential effect of oxpentifylline on tumour necrosis factor and interleukin-6 production. Lancet, 1990. 335(8688): p. 543.

48. Barton, B.E., et al., Cytokine inhibition by a novel steroid, mometasone furoate. Immunopharmacol Immunotoxicol, 1991. 13(3): p. 251-61.

49. Kutteh, W.H., W.E. Rainey, and B.R. Carr, Glucocorticoids inhibit lipopolysaccharide-induced production of tumor necrosis factor-alpha by human fetal Kupffer cells. J Clin Endocrinol Metab, 1991. 73(2): p. 296-301.

50. Allen, J.N., D.J. Herzyk, and M.D. Wewers, Colchicine has opposite effects on interleukin-1 beta and tumor necrosis factor-alpha production. Am J Physiol, 1991. 261(4 Pt 1): p. L315-21.

51. Jeong, J.Y. and D.M. Jue, Chloroquine inhibits processing of tumor necrosis factor in lipopolysaccharide-stimulated RAW 264.7 macrophages. J Immunol, 1997. 158(10): p. 4901-7.

52. Kawano, K., et al., FK 506 ameliorates normothermic liver ischemia in rats by suppressing production of tumor necrosis factor. Transpl Int, 1992. 5 Suppl 1: p. S665-9.

53. Kawano, K., et al., Evidence that FK506 alleviates ischemia/reperfusion injury to the rat liver: in vivo demonstration for suppression of TNF-a production in response to endotoxemia. Eur Surg Res, 1994. 26(2): p. 108-15.

54. Smith, C.S., et al., Cyclosporin A blocks induction of tumor necrosis factor-alpha in human B lymphocytes. Biochem Biophys Res Commun, 1994. 204(1): p. 383-90.

55. Molnar-Kimber, K.L., et al., Differential regulation of TNF-alpha and IL-1beta production from endotoxin stimulated human monocytes by phosphodiesterase inhibitors. Mediators Inflamm, 1992. 1(6): p. 411-7.

56. Wang, M.H., et al., Inhibition of endotoxin or lipid A-induced tumor necrosis factor production by synthetic lipid A partial structures in human peripheral blood mononuclear cells. Lymphokine Cytokine Res, 1992. 11(1): p. 23-31.

57. Molnar-Kimber, K., et al., Modulation of TNF alpha and IL-1 beta from endotoxin-stimulated monocytes by selective PDE isozyme inhibitors. Agents Actions, 1993. 39 Spec No: p. C77-9.

58. Giroir, B.P. and B. Beutler, Effect of amrinone on tumor necrosis factor production in endotoxic shock. Circ Shock, 1992. 36(3): p. 200-7.

59. Peristeris, P., et al., N-acetylcysteine and glutathione as inhibitors of tumor necrosis factor production. Cell Immunol, 1992. 140(2): p. 390-9.

60. Schreck, R., et al., Dithiocarbamates as potent inhibitors of nuclear factor kappa B activation in intact cells. J Exp Med, 1992. 175(5): p. 1181-94.

61. Thomassen, M.J., et al., Synthetic surfactant (Exosurf) inhibits endotoxin-stimulated cytokine secretion by human alveolar macrophages. Am J Respir Cell Mol Biol, 1992. 7(3): p. 257-60.

62. Danenberg, H.D., et al., Dehydroepiandrosterone protects mice from endotoxin toxicity and reduces tumor necrosis factor production. Antimicrob Agents Chemother, 1992. 36(10): p. 2275-9.

63. Zheng, Z.M., S. Specter, and H. Friedman, Serum proteins affect the inhibition by delta-tetrahydrocannabinol of tumor necrosis factor alpha production by mouse macrophages. Adv Exp Med Biol, 1993. 335: p. 89-93.

64. Fischer-Stenger, K., D.A. Dove Pettit, and G.A. Cabral, Delta 9-tetrahydrocannabinol inhibition of tumor necrosis factor-alpha: suppression of post-translational events. J Pharmacol Exp Ther, 1993. 267(3): p. 1558-65.

65. Monastra, G., et al., Phosphatidylserine, a putative inhibitor of tumor necrosis factor, prevents autoimmune demyelination. Neurology, 1993. 43(1): p. 153-63.

66. Ogata, M., et al., An antagonist of platelet-activating factor suppresses endotoxin-induced tumor necrosis factor and mortality in mice pretreated with carrageenan. Infect Immun, 1993. 61(2): p. 699-704.

67. Moreira, A.L., et al., Thalidomide exerts its inhibitory action on tumor necrosis factor alpha by enhancing mRNA degradation. J Exp Med, 1993. 177(6): p. 1675-80.

68. Tramontana, J.M., et al., Thalidomide treatment reduces tumor necrosis factor alpha production and enhances weight gain in patients with pulmonary tuberculosis. Mol Med, 1995. 1(4): p. 384-97.

69. Schmidt, H., et al., Thalidomide inhibits TNF response and increases survival following endotoxin injection in rats. J Surg Res, 1996. 63(1): p. 143-6.

70. Fantuzzi, G., et al., Inhibitors of cytochrome P450 suppress tumor necrosis factor production. Cell Immunol, 1993. 150(2): p. 417-24.

71. Shinji, H., K.S. Akagawa, and T. Yoshida, Cytochalasin D inhibits lipopolysaccharide-induced tumor necrosis factor production in macrophages. J Leukoc Biol, 1993. 54(4): p. 336-42.

72. Takenaka, I., et al., Ketamine suppresses endotoxin-induced tumor necrosis factor alpha production in mice. Anesthesiology, 1994. 80(2): p. 402-8.

73. Stevens, D.B., K.E. Gould, and R.H. Swanborg, Transforming growth factor-beta 1 inhibits tumor necrosis factor-alpha/lymphotoxin production and adoptive transfer of disease by effector cells of autoimmune encephalomyelitis. J Neuroimmunol, 1994. 51(1): p. 77-83.

74. Wang, P., et al., Interleukin (IL)-10 inhibits nuclear factor kappa B (NF kappa B) activation in human monocytes. IL-10 and IL-4 suppress cytokine synthesis by different mechanisms. J Biol Chem, 1995. 270(16): p. 9558-63.

75. Bernard, C., et al., Pentoxifylline selectivity inhibits tumor necrosis factor synthesis in the arterial wall. J Cardiovasc Pharmacol, 1995. 25 Suppl 2: p. S30-3.

76. Kaplan, J.M., et al., Effect of TNF alpha production inhibitors BRL 61063 and pentoxifylline on the response of rats to poly I:C. Toxicology, 1995. 95(1-3): p. 187-96.

77. Hotchkiss, R.S., et al., Calcium antagonists decrease plasma and tissue concentrations of tumor necrosis factor-alpha, interleukin-1 beta, and interleukin-1 alpha in a mouse model of endotoxin. Shock, 1995. 3(5): p. 337-42.

78. Chan, M.M., Inhibition of tumor necrosis factor by curcumin, a phytochemical. Biochem Pharmacol, 1995. 49(11): p. 1551-6.

79. Belkowski, S.M., et al., Inhibition of interleukin-1 and tumor necrosis factor-alpha synthesis following treatment of macrophages with the kappa opioid agonist U50, 488H. J Pharmacol Exp Ther, 1995. 273(3): p. 1491-6.

80. Sansoni, P., et al., Inhibition of antigen -presenting cell function by alendronate in vitro. J Bone Miner Res, 1995. 10(11): p. 1719-25.

81. Onai, N., et al., Inhibitory effects of bisbenzylisoquinoline alkaloids on induction of proinflammatory cytokines, interleukin-1 and tumor necrosis factor-alpha. Planta Med, 1995. 61(6): p. 497-501.

82. Ferrante, A., et al., Tetrandrine, a plant alkaloid, inhibits the production of tumour necrosis factor-alpha (cachectin) hy human monocytes. Clin Exp Immunol, 1990. 80(2): p. 232-5.

83. Bissonnette, E.Y., J.A. Enciso, and A.D. Befus, Inhibitory effects of sulfasalazine and its metabolites on histamine release and TNF-alpha production by mast cells. J Immunol, 1996. 156(1): p. 218-23.

84. van der Poll, T., et al., Epinephrine inhibits tumor necrosis factor-alpha and potentiates interleukin 10 production during human endotoxemia. J Clin Invest, 1996. 97(3): p. 713-9.

85. Warr, G.A., et al., BMS-182123, a fungal metabolite that inhibits the production of TNF-alpha by macrophages and monocytes. J Antibiot (Tokyo), 1996. 49(3): p. 234-40.

86. Sajjadi, F.G., et al., Inhibition of TNF-alpha expression by adenosine: role of A3 adenosine receptors. J Immunol, 1996. 156(9): p. 3435-42.

87. Eigler, A., et al., Endogenous adenosine curtails lipopolysaccharide-stimulated tumour necrosis factor synthesis. Scand J Immunol, 1997. 45(2): p. 132-9.

88. Goto, M., et al., Inhibitory effect of E3330, a novel quinone derivative able to suppress tumor necrosis factor-alpha generation, on activation of nuclear factor-kappa B. Mol Pharmacol, 1996. 49(5): p. 860-73.

89. Li, Q., et al., Nicotine reduces TNF-alpha expression through a alpha7 nAChR/MyD88/NF-kB pathway in HBE16 airway epithelial cells. Cell Physiol Biochem, 2011. 27(5): p. 605-12.

90. Madretsma, G.S., et al., Nicotine inhibits the in vitro production of interleukin 2 and tumour necrosis factor-alpha by human mononuclear cells. Immunopharmacology, 1996. 35(1): p. 47-51.

91. Toungouz, M., C. Denys, and E. Dupont, Blockade of proliferation and tumor necrosis factor-alpha production occurring during mixed lymphocyte reaction by interferon-gamma-specific natural antibodies contained in intravenous immunoglobulins. Transplantation, 1996. 62(9): p. 1292-6.

92. Darville, T., L.B. Milligan, and K.K. Laffoon, Intravenous immunoglobulin inhibits staphylococcal toxin-induced human mononuclear phagocyte tumor necrosis factor alpha production. Infect Immun, 1997. 65(2): p. 366-72.

93. Benigni, F., et al., Cardiotrophin-1 inhibits tumor necrosis factor production in the heart and serum of lipopolysaccharide-treated mice and in vitro in mouse blood cells. Am J Pathol, 1996. 149(6): p. 1847-50.

94. Feng, Y, et al., Inhibition of LPS-induced TNF-alpha production by calcitonin gene-related peptide (CGRP) in cultured mouse peritoneal macrophages. Life Sci, 1997. 61(20): p. PL 281-7.

95. Qiu, H.B., et al., Effects of dexamethasone, ibuprofen, and ligustrazini on lipopolysaccharides-induced tumor necrosis factor alpha production. Zhongguo Yao Li Xue Bao, 1997. 18(1): p. 67-70.

96. Shohami, E., et al., Cytokine production in the brain following closed head injury: dexanabinol (HU-211) is a novel TNF-alpha inhibitor and an effective neuroprotectant. J Neuroimmunol, 1997. 72(2): p. 169-77.

97. Jorres, A., et al., Inhibition of tumour necrosis factor production in endotoxin-stimulated human mononuclear leukocytes by the prostacyclin analogue iloprost: cellular mechanisms. Cytokine, 1997. 9(2): p. 119-25.

98. Murakami, K., et al., Activated protein C prevents LPS-induced pulmonary vascular injury by inhibiting cytokine production. Am J Physiol, 1997. 272(2 Pt 1): p. L197-202.

99. Thissen, J.P. and J. Verniers, Inhibition by interleukin-1 beta and tumor necrosis factor-alpha of the insulin-like growth factor I messenger ribonucleic acid response to growth hormone in rat hepatocyte primary culture. Endocrinology, 1997. 138(3): p. 1078-84.

100. Zhang, M., et al., Spermine inhibits proinflammatory cytokine synthesis in human mononuclear cells: a counterregulatory mechanism that restrains the immune response. J Exp Med, 1997. 185(10): p. 1759-68.

101. Yamamoto, N., et al., FR167653, a dual inhibitor of interleukin-1 and tumor necrosis factor-alpha, ameliorates endotoxin-induced shock. Eur J Pharmacol, 1997. 327(2-3): p. 169-74.

102. Solorzano, C.C., et al., A matrix metalloproteinase inhibitor prevents processing of tumor necrosis factor alpha (TNF alpha) and abrogates endotoxin-induced lethality. Shock, 1997. 7(6): p. 427-31.

103. Deshpande, R., et al., Estradiol down-regulates LPS-induced cytokine production and NFkB activation in murine macrophages. Am J Reprod Immunol, 1997. 38(1): p. 46-54.

104. Shackelford, R.E., et al., Aspirin inhibits tumor necrosis factoralpha gene expression in murine tissue macrophages. Mol Pharmacol, 1997. 52(3): p. 421-9.

105. Matsumori, A., et al., Amiodarone inhibits production of tumor necrosis factor-alpha by human mononuclear cells: a possible mechanism for its effect in heart failure. Circulation, 1997. 96(5): p. 1386-9.

106. Kettelhut, I.C., W. Fiers, and A.L. Goldberg, The toxic effects of tumor necrosis factor in vivo and their prevention by cyclooxygenase inhibitors. Proc Natl Acad Sci U S A, 1987. 84(12): p. 4273-7.

107. Rand, T.H., et al., Nedocromil sodium and cromolyn (sodium cromoglycate) selectively inhibit antibody-dependent granulocyte-mediated cytotoxicity. Int Arch Allergy Appl Immunol, 1988. 87(2): p. 151-8.

108. Lin, Y., P.G. Case, and P.Q. Patek, Inhibition of tumour necrosis factor and natural cytotoxic cell lytic activities by a spleen cell-elaborated factor. Immunology, 1988. 63(4): p. 663-8.

109. Sullivan, G.W., et al., Inhibition of the inflammatory action of interleukin-1 and tumor necrosis factor (alpha) on neutrophil function by pentoxifylline. Infect Immun, 1988. 56(7): p. 1722-9.

110. Gibson, R.L., et al., Group B streptococcus induces tumor necrosis factor in neonatal piglets. Effect of the tumor necrosis factor inhibitor pentoxifylline on hemodynamics and gas exchange. Am Rev Respir Dis, 1991. 143(3): p. 598-604.

111. Kozaki, K., et al., Pentoxifylline inhibits production of superoxide anion and tumor necrosis factor by Kupffer cells in rat liver preservation. Transplant Proc, 1993. 25(6): p. 3025-6.

112. Seckinger, P., et al., Tumor necrosis factor inhibitor: purification, NH2-terminal amino acid sequence and evidence for anti-inflammatory and immunomodulatory activities. Eur J Immunol, 1990. 20(5): p. 1167-74.

113. Kilbourn, R.G., et al., NG-methyl-L-arginine inhibits tumor necrosis factor-induced hypotension: implications for the involvement of nitric oxide. Proc Natl Acad Sci U S A, 1990. 87(9): p. 3629-32.

114. Mayoral, J.L. and D.L. Dunn, Cross-reactive murine monoclonal antibodies directed against the core/lipid A region of endotoxin inhibit production of tumor necrosis factor. J Surg Res, 1990. 49(4): p. 287-92.

115. Mukaida, N., et al., Dexamethasone inhibits the induction of monocyte chemotactic-activating factor production by IL-1 or tumor necrosis factor. J Immunol, 1991. 146(4): p. 1212-5.

116. Gadina, M., et al., Protective effect of chlorpromazine on endotoxin toxicity and TNF production in glucocorticoid-sensitive and glucocorticoid-resistant models of endotoxic shock. J Exp Med, 1991. 173(6): p. 1305-10.

117. LaMarre, J., et al., Cytokine binding and clearance properties of proteinase-activated alpha 2-macroglobulins. Lab Invest, 1991. 65(1): p. 3-14.

118. Shainkin-Kestenbaum, R., et al., Acute phase protein, serum amyloid A, inhibits IL-1- and TNF-inducedfever and hypothalamic PGE2 in mice. Scand J Immunol, 1991. 34(2): p. 179-83.

119. van Kessel, K.P., J.A. van Strijp, and J. Verhoef, Inactivation of recombinant human tumor necrosis factor-alpha by proteolytic enzymes released from stimulated human neutrophils. J Immunol, 1991. 147(11): p. 3862-8.

120. Bagby, G.J., et al., Attenuation of glucose metabolic changes resulting from TNF-alpha administration by adrenergic blockade. Am J Physiol, 1992. 262(4 Pt 2): p. R628-35.

121. Ogata, M., et al., Protective effects of a leukotriene inhibitor and a leukotriene antagonist on endotoxin-induced mortality in carrageenan-pretreated mice. Infect Immun, 1992. 60(6): p. 2432-7.

122. Parry, E.W., Cycloheximide or nordihydroguaiaretic acid protects mice against the lethal and hepatocytolytic effects of a combined challenge with D-galactosamine and bacterial endotoxin. J Comp Pathol, 1993. 108(2): p. 185-90.

123. van Hinsbergh, V.W., et al., Genistein reduces tumor necrosis factor alpha-induced plasminogen activator inhibitor-1 transcription but not urokinase expression in human endothelial cells. Blood, 1994. 84(9): p. 2984-91.

124. Taniguchi, S., et al., Butylated hydroxyanisole blocks the inhibitory effects of tumor necrosis factor-alpha on collagen production in human dermal fibroblasts. J Dermatol Sci, 1996. 12(1): p. 44-9.

125. Cohen, P.S., et al., CNI-1493 inhibits monocyte/macrophage tumor necrosis factor by suppression of translation efficiency. Proc Natl Acad Sci U S A, 1996. 93(9): p. 3967-71.

126. Zhang, J.P., et al., Inhibitory effect of quercetin on tumor necrosis factor and interleukin-1 beta pro-osteoclastic activities. Zhongguo Yao Li Xue Bao, 1996. 17(3): p. 261-3.

127. Murakami, K., et al., Gabexate mesilate, a synthetic protease inhibitor, attenuates endotoxin-induced pulmonary vascular injury by inhibiting tumor necrosis factor production by monocytes. Crit Care Med, 1996. 24(6): p. 1047-53.

128. Murakami, K., et al., A novel platelet activating factor antagonist, SM-12502, attenuates endotoxin-induced disseminated intravascular coagulation and acute pulmonary vascular injury by inhibiting TNF production in rats. Thromb Haemost, 1996. 75(6): p. 965-70.

129. Suzuki, K., et al., Monoclonal nonspecific suppressor factor beta (MNSF beta) inhibits the production of TNF-alpha by lipopolysaccharide-activated macrophages. Immunobiology, 1996. 195(2): p. 187-98.

130. Munoz, C., et al., Pyrrolidine dithiocarbamate inhibits the production of interleukin-6, interleukin-8, and granulocyte-macrophage colony-stimulating factor by human endothelial cells in response to inflammatory mediators: modulation of NF-kappa B and AP-1 transcription factors activity. Blood, 1996. 88(9): p. 3482-90.

131. Hill, G.E. and R.A. Robbins, Aprotinin but not tranexamic acid inhibits cytokine-induced inducible nitric oxide synthase expression. Anesth Analg, 1997. 84(6): p. 1198-202.

132. Yamato, K., Z. el-Hajjaoui, and H.P. Koeffler, Regulation of levels of IL-1 mRNA in human fibroblasts. J Cell Physiol, 1989. 139(3): p. 610-6.

133. Zucali, J.R., C. Morse, and C.A. Dinarello, The role of protein kinase C in interleukin 1 and tumor necrosis factor alpha induction of fibroblasts to produce and release granulocyte-macrophage colony-stimulating activity. Exp Hematol, 1990. 18(8): p. 888-92.

134. Hori, T., et al., Prostaglandins antagonize fibroblast proliferation stimulated by tumor necrosis factor. Biochem Biophys Res Commun, 1991. 174(2): p. 758-66.

What is claimed is:

1. A method of inhibiting and/or reversing blood vessel degeneration and/or promoting blood vessel regeneration in an individual, the method comprising the step of administering to the individual a therapeutically effective amount of a composition comprising a fibroblast cell population, fibroblast-derived products, and/or conditioned media therefrom.

2. The method of claim 1, wherein the fibroblast cell population is obtained from blood, placenta, bone marrow, amniotic fluid, amniotic membrane, circulating fibroblasts, testicular tissues, adipose tissue, exfoliated teeth, hair follicle, dermal tissue, side population cells, or a combination thereof.

3. The method of claim 1 or 2, wherein the fibroblast cell population is autologous, allogeneic, xenogeneic, and a mixture thereof.

4. The method of any one of claims 1-3, wherein the fibroblast cell population is derived from a donor younger in age than a recipient.

5. The method of any one of claims 1-4, wherein the fibroblasts are CD73-positive and/or CD56-positive fibroblasts.

6. The method of any one of claims 1-5, wherein the individual has at least one aneurysm.

7. The method of claim 6, wherein the aneurysm is an aortic aneurysm or saccular aneurysm.

8. The method of any of claims 1-7, wherein one or more additional therapeutic agents and/or conditions are administered in combination with the fibroblast cell population.

9. The method of claim 8, wherein the one or more additional therapeutic agents are capable of: a) stimulating fibroblast integration into the blood vessels; b) augmenting regenerative activity of endogenous and/or exogenous fibroblasts; c) mobilizing endothelial progenitor cells; d) stimulating smooth muscle cell proliferation; e) inducing nitric oxide activity; or f) a combination thereof.

10. The method of claim 9, wherein the one or more additional therapeutic agents capable of stimulating fibroblast cell integration into parts of blood vessels is selected from the group consisting of a matrix metalloprotease inhibitor, an antioxidant, a chemoattractant, and combinations thereof.

11. The method of claim 9, wherein the one or more additional therapeutic agents capable of stimulating fibroblast regenerative cell activity is selected from the group consisting of erythropoietin, human chorionic gonadotrophin, parathyroid hormone, G-CSF, GM-CSF, valproic acid, thalidomide, sodium phenybutyrate, FGF-1, FGF-2, and combinations thereof.

12. The method of claim 9, wherein the one or more additional therapeutic agents capable of mobilizing endothelial progenitor cells is selected from the group consisting of G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, and combinations thereof.

13. The method of claim 9, wherein the one or more additional therapeutic conditions capable of mobilizing endothelial progenitor cells comprises exposure to one or more conditions sufficient to mobilize endothelial progenitor cells.

14. The method of claim 13, wherein the one or more conditions are selected from the group consisting of exercise, hyperbaric oxygen, autohemotherapy by ex vivo ozonation of peripheral blood, induction of SDF-1 secretion in an anatomical area outside of the bone marrow, and combinations thereof.

15. The method of claim 9, wherein the one or more additional therapeutic agents capable of stimulating smooth muscle proliferation is selected from the group consisting of PDGF-1, PDGF-BB, BTC-GF, estradiol, and combinations thereof.

16. The method of claim 9, wherein the one or more additional therapeutic agents inductive of nitric oxide activity is selected from the group consisting of lipoteichoic acid, cinnamic acid, resveratrol, FGF, and combinations thereof.

17. The method of any of claims 1-16, wherein the fibroblast cell population is administered intravenously.

18. The method of any of claims 1-17, wherein the fibroblast cell population is administered once every other day.

19. The method any one of claims 1-18, wherein the fibroblast cell population is administered once every other day over the course of 7 days.

20. The method of any one of claims 1-19, wherein administration of the fibroblast cell population inhibits inflammation and/or accelerates re-endothelialization.

21. A method of treating one or more aneurysms in an individual, the method comprising the step of administering to the individual a therapeutically effective amount of a composition comprising one or more cell populations.

22. The method of claim 21, wherein the one or more cell populations are administered intravenously.

23. The method claim 21 or 22, wherein the one or more cell populations are administered once every other day.

24. The method any one of claims 21-23, wherein the one or more cell populations are administered once every other day over the course of 7 days.

25. The method of any one of claims 21-24, wherein the one or more cell populations are administered in sequence or at the same time.

26. The method of any one of claims 21-25, wherein administration of the one or more cell populations induces an environment conducive for blood vessel regeneration, restoration of blood vessel function, and/or reversal of blood vessel degeneration.

27. The method of claim 26, wherein the environment is associated with reduced fibrosis, enhanced growth factor production, and stimulation of cellular proliferation.

28. The method of claim 26 or 27, wherein blood vessel regeneration, restoration of blood vessel function, and/or reversal of blood vessel degeneration extends the life of a mammal as a result of appropriate production of anti-coagulating/clotting factors, control of angiogenesis, appropriate revascularization of injured tissue, decrease in age-related atherosclerosis, and prevention of loss of anti-thrombotic activity of endothelium associated with age.

29. The method of any one of claims 21-28, wherein the one or more cell populations comprise fibroblast cells and/or stem cells.

30. The method of claim 29, wherein at least one cell population comprises CD73-positive fibroblasts and/or CD56-positive fibroblasts administered individually or together.

31. The method of claim 29 or 30, wherein the fibroblast cells are obtained from cord blood, placenta, Wharton’s jelly, circulating peripheral blood, adipose tissue derived, exfoliated teeth, hair follicle, dermis, menstrual blood, endometrium, amnion, amniotic fluid, or a combination thereof.

32. The method of claim 30 or 31, wherein approximately 1-500 million CD73-positive and/or CD56-positive fibroblast cells are administered to the individual.

33. The method of claim 32, wherein approximately 750,000-1,250,000 CD73-positive and/or CD56-positive fibroblast cells are administered to the individual.

34. The method of any of claims 29-33, wherein the stem cells are selected from the group consisting of hematopoietic stem cells, embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells, side population stem cells, and combinations thereof.

35. The method of any one of claims 21-34, wherein the composition further comprises a fibroblast cell activator.

36. The method of claim 35, wherein the activator is selected from the group consisting of erythropoietin, human chorionic gonadotrophin, parathyroid hormone, G-CSF, GM-CSF, valproic acid, thalidomide, sodium phenybutyrate, FGF-1, FGF-2, and combinations thereof.

37. The method of claim 35 or 36, wherein the fibroblast cell activator increases the therapeutic activity of fibroblasts.

38. The method of claim 37, wherein the therapeutic activity comprises increased production of FGF-1, anti-inflammation, stimulation of tissue regeneration, or combinations thereof.

39. The method of any of claims 21-38, wherein one or more additional therapeutic agents are administered in combination with the one or more cell populations.

40. The method of claim 39, wherein the one or more additional therapeutic agents are capable of: a) stimulating fibroblast integration into the blood vessels; b) augmenting regenerative activity of endogenous and/or exogenous fibroblasts; c) stimulating smooth muscle cell proliferation; d) inducing nitric oxide activity; or e) a combination thereof.

41. The method of claim 40, wherein the one or more additional therapeutic agents capable of stimulating fibroblast cell integration into parts of blood vessels is selected from the group consisting of a matrix metalloprotease inhibitor, an antioxidant, a chemoattractant, and combinations thereof.

42. The method of claim 40, wherein the one or more additional therapeutic agents capable of stimulating fibroblast regenerative cell activity is selected from the group consisting of erythropoietin, human chorionic gonadotrophin, parathyroid hormone, G-CSF, GM-CSF, valproic acid, thalidomide, sodium phenybutyrate, FGF-1, FGF-2, and combinations thereof.

43. The method of claim 40, wherein the one or more additional therapeutic agents capable of stimulating smooth muscle proliferation is selected from the group consisting of PDGF-1, PDGF-BB, BTC-GF, estradiol, and combinations thereof.

44. The method of claim 40, wherein the one or more additional therapeutic agents inductive of nitric oxide activity is selected from the group consisting of lipoteichoic acid, cinnamic acid, resveratrol, FGF, and combinations thereof.

45. The method of any one of claims 21-44, further comprising mobilization of endogenous endothelial progenitor cells from the bone marrow.

46. The method of claim 45, wherein mobilization is achieved by administration of an agent selected from the group consisting of G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA)reductase inhibitors, small molecule antagonists of SDF-1, and combinations thereof.

47. The method of claim 45, wherein mobilization is achieved by exposure to one or more conditions sufficient to mobilize endothelial progenitor cells.

48. The method of claim 47, wherein the one or more conditions are selected from the group consisting of exercise, hyperbaric oxygen, autohemotherapy by ex vivo ozonation of peripheral blood, induction of SDF-1 secretion in an anatomical area outside of the bone marrow, and combinations thereof.

49. A method of treating one or more aneurysms in an individual, the method comprising the step of administering to the individual a therapeutically effective amount of a composition comprising at least one fibroblast and/or stem cell population.

50. The method of claim 49, wherein the at least one stem cell population is selected from the group consisting of hematopoietic stem cells, embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells, side population stem cells, and combinations thereof.

51. The method of claim 49 or 50, wherein the at least one fibroblast cell population is obtained from cord blood, placenta, wharton’s jelly, circulating peripheral blood, adipose tissue derived, exfoliated teeth, hair follicle, dermis, menstrual blood, endometrium, amnion, amniotic fluid, or a combination thereof.

52. The method of any of claims 49-51, wherein the at least one fibroblast and/or stem cell population is administered at a concentration ranging from about 500,000 to about 200 million cells.

53. The method of claim 52, wherein the at least one fibroblast and/or stem cell population is administered at a concentration ranging from about 750,000 to about 1,250,000 cells.

54. The method of any of claims 49-53, wherein the at least one fibroblast and/or stem cell population is administered intravenously.

55. The method any of claims 49-54, wherein the one or more cell populations are administered once every other day.

56. The method any one of claims 49-55, wherein the one or more cell populations are administered once every other day over the course of 7 days.

57. The method of any one of claims 49-56, wherein the one or more cell populations are administered in sequence or at the same time.

58. The method of any of claims 49-57, wherein the aneurysm is an aortic aneurysm or a saccular aneurysm.

59. The method of claim 58, wherein said saccular aneurysm is located in the brain.

60. The method of any of claims 49-59, wherein the fibroblast cell population inhibits progression or induces regression of an aneurysm.

61. The method of claim 60, wherein inhibition of progression of an aneurysm comprises suppression of progressive blood vessel weakening.

62. The method of claim 60, wherein induction of regression of an aneurysm comprises a reduction in circumference of an aneurysmic blood vessel.

63. The method of any of claims 49-62, wherein the at least one fibroblast and/or stem cell population expresses the markers C56, CD90, and/or CD105, and wherein administration of the at least one fibroblast and/or stem cell population inhibits progression of an aortic aneurysm.

64. The method of claim 63, wherein the at least one fibroblast and/or stem cell population lacks expression of CD34 and/or CD45.

65. The method of any of claims 49-62, wherein a CD56-positive fibroblast cell population is administered together with a mesenchymal or mesenchymal-like stem cell population, and wherein progression of an aortic aneurysm is inhibited.

66. The method of any of claims 49-62, wherein the at least one fibroblast and/or stem cell population expresses the markers CD90 and CD105, and wherein administration of the at least one fibroblast and/or stem cell population induces regression of an aortic aneurysm.

67. The method of claim 66, wherein the at least one fibroblast and/or stem cell population lacks expression of CD34 and CD45.

68. The method of any of claims 49-62, wherein a CD73-positive fibroblast cell population is administered together with a mesenchymal or mesenchymal-like stem cell population, and wherein regression of an aortic aneurysm is induced.

69. The method of any of claims 49-62, wherein the at least one fibroblast and/or stem cell population expresses the markers CD56, CD90, and CD105, and wherein administration of the at least one fibroblast and/or stem cell population inhibits progression of a saccular aneurysm.

70. The method of claim 69, wherein the at least one fibroblast and/or stem cell population lacks expression of CD34 and CD45.

71. The method of any of claims 49-62, wherein a CD73-positive fibroblast cell population is administered together with a mesenchymal or mesenchymal-like stem cell population, and wherein progression of a saccular aneurysm is inhibited.

72. The method of any of claims 49-62, wherein the at least one fibroblast and/or stem cell population expresses the markers CD56, CD90, and CD105, and wherein administration of the at least one fibroblast and/or stem cell population induces regression of a saccular aneurysm.

73. The method of claim 72, where the at least one fibroblast and/or stem cell population lacks expression of CD34 and CD45.

74. The method of any of claims 49-62, wherein a CD56-positive fibroblast cell population is administered together with a mesenchymal or mesenchymal-like stem cell population, and wherein regression of a saccular aneurysm is induced.

75. The method of any of claims 49-74, wherein one or more additional therapeutic agents and/or conditions are administered in combination with the at least one fibroblast and/or stem cell population.

76. The method of claim 75, wherein the one or more additional therapeutic agents are capable of: a) stimulating fibroblast integration into the blood vessels; b) augmenting regenerative activity of endogenous and/or exogenous fibroblasts; c) mobilizing endothelial progenitor cells; d) stimulating smooth muscle cell proliferation; e) inducing nitric oxide activity; or f) a combination thereof.

77. The method of claim 76, wherein the one or more additional therapeutic agents capable of stimulating fibroblast cell integration into parts of blood vessels is selected from the group consisting of a matrix metalloprotease inhibitor, an antioxidant, a chemoattractant, and combinations thereof.

78. The method of claim 76, wherein the one or more additional therapeutic agents capable of stimulating fibroblast regenerative cell activity is selected from the group consisting of erythropoietin, human chorionic gonadotrophin, parathyroid hormone, G-CSF, GM-CSF, valproic acid, thalidomide, sodium phenybutyrate, FGF-1, FGF-2, and combinations thereof.

79. The method of claim 76, wherein the one or more additional therapeutic agents capable of mobilizing endothelial progenitor cells is selected from the group consisting of G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, and combinations thereof.

80. The method of claim 76, wherein the one or more additional therapeutic conditions capable of mobilizing endothelial progenitor cells comprises exposure to one or more conditions sufficient to mobilize endothelial progenitor cells.

81. The method of claim 80, wherein the one or more conditions are selected from the group consisting of exercise, hyperbaric oxygen, autohemotherapy by ex vivo ozonation of peripheral blood, induction of SDF-1 secretion in an anatomical area outside of the bone marrow, and combinations thereof.

82. The method of claim 76, wherein the one or more additional therapeutic agents capable of stimulating smooth muscle proliferation is selected from the group consisting of PDGF-1, PDGF-BB, BTC-GF, estradiol, and combinations thereof.

83. The method of claim 76, wherein the one or more additional therapeutic agents inductive of nitric oxide activity is selected from the group consisting of lipoteichoic acid, cinnamic acid, resveratrol, FGF, and combinations thereof.

84. A method of inhibiting development of aortic dissection and/or reversing blood flow abnormalities associated with aortic dissection, the method comprising the step of administering to the individual a therapeutically effective amount of a composition comprising at least one fibroblast and/or stem cell population.

85. The method of claim 84, wherein the at least one stem cell population is selected from the group consisting of hematopoietic stem cells, embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells, side population stem cells, and combinations thereof.

86. The method of claim 84 or 85, wherein the at least one fibroblast population is obtained from cord blood, placenta, wharton’s jelly, circulating peripheral blood, adipose tissue derived, exfoliated teeth, hair follicle, dermis, menstrual blood, endometrium, amnion, amniotic fluid, or a combination thereof.

87. The method of any of claims 84-86, wherein the at least one fibroblast and/or stem cell population is administered at a concentration ranging from about 500,000 to about 200 million cells.

88. The method of claim 87, wherein the at least one fibroblast and/or stem cell population is administered at a concentration ranging from about 750,000 to about 1,250,000 cells.

89. The method of any of claims 84-88, wherein the at least one fibroblast and/or stem cell population is administered intravenously.

90. The method any of claims 84-89, wherein the one or more cell populations are administered once every other day.

91. The method any one of claims 84-90, wherein the one or more cell populations are administered once every other day over the course of 7 days.

92. The method of any one of claims 84-91, wherein the one or more cell populations are administered in sequence or at the same time.

93. The method of any of claims 84-92, wherein administration of the at least one fibroblast and/or stem cell population strengthens the aorta intimal layer, thereby reducing the probability of tears in the intimal layer.

94. The method of any of claims 84-93, wherein administration of the at least one fibroblast and/or stem cell population reduces accumulation of basophilic ground substances and the extent of medial cystic necrosis.

95. The method of any of claims 84-94, wherein administration of the at least one fibroblast and/or stem cell population restores substantially normal blood flow.

96. The method of any of claims 84-95, wherein the at least one fibroblast and/or stem cell population expresses the markers CD56, CD90, and CD105, and wherein administration of the at least one fibroblast and/or stem cell population inhibits development of aortic dissection.

97. The method of claim 96, wherein the at least one fibroblast and/or stem cell population lacks expression of CD34 and CD45.

98. The method of any of claims 84-95, wherein a CD56-positive fibroblast cell population is administered together with a mesenchymal or mesenchymal-like stem cell population, and wherein development of aortic dissection is inhibited.

99. The method of any of claims 84-95, wherein the at least one fibroblast and/or stem cell population expresses the markers CD56, CD90, and CD105, and wherein administration of the at least one fibroblast and/or stem cell population reverses blood flow abnormalities.

100. The method of claim 99, wherein the at least one fibroblast and/or stem cell population lacks expression of CD34 and CD45.

101. The method of any of claims 84-95, wherein a CD73-positive stem cell population is administered together with a mesenchymal or mesenchymal-like stem cell population, and wherein blood flow abnormalities are reversed.

102. The method of any of claims 84-101, wherein one or more additional therapeutic agents and/or conditions are administered in combination with the at least one fibroblast and/or stem cell population.

103. The method of claim 102, wherein the one or more additional therapeutic agents are capable of: a) stimulating fibroblast integration into the blood vessels; b) augmenting regenerative activity of endogenous and/or exogenous fibroblasts; c) mobilizing endothelial progenitor cells; d) stimulating smooth muscle cell proliferation; e) inducing nitric oxide activity; or f) a combination thereof.

104. The method of claim 103, wherein the one or more additional therapeutic agents capable of stimulating fibroblast cell integration into parts of blood vessels is selected from the group consisting of a matrix metalloprotease inhibitor, an antioxidant, a chemoattractant, and combinations thereof.

105. The method of claim 103, wherein the one or more additional therapeutic agents capable of stimulating fibroblast regenerative cell activity is selected from the group consisting of erythropoietin, human chorionic gonadotrophin, parathyroid hormone, G-CSF, GM-CSF, valproic acid, thalidomide, sodium phenybutyrate, FGF-1, FGF-2, and combinations thereof.

106. The method of claim 103, wherein the one or more additional therapeutic agents capable of mobilizing endothelial progenitor cells is selected from the group consisting of G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, and combinations thereof.

107. The method of claim 103, wherein the one or more additional therapeutic conditions capable of mobilizing endothelial progenitor cells comprises exposure to one or more conditions sufficient to mobilize endothelial progenitor cells.

108. The method of claim 107, wherein the one or more conditions are selected from the group consisting of exercise, hyperbaric oxygen, autohemotherapy by ex vivo ozonation of peripheral blood, induction of SDF-1 secretion in an anatomical area outside of the bone marrow, and combinations thereof.

109. The method of claim 103, wherein the one or more additional therapeutic agents capable of stimulating smooth muscle proliferation is selected from the group consisting of PDGF-1, PDGF-BB, BTC-GF, estradiol, and combinations thereof.

110. The method of claim 103, wherein the one or more additional therapeutic agents inductive of nitric oxide activity is selected from the group consisting of lipoteichoic acid, cinnamic acid, resveratrol, FGF, and combinations thereof.
